(19) United States
(12) Patent Application Publication
(10) Pub. No.: US 2026/0151539 A1
(43) Pub. Date: Jun. 4, 2026

(54) INJECTABLE RECOMBINANT PROTEIN-BASED HYDROGELS FOR THERAPEUTIC DELIVERY

(71) Applicant: University of Washington, Seattle, WA (US)

(72) Inventors: Nicole Gregorio, Seattle, WA (US); Cole DeForest, Seattle, WA (US); Mary O'Kelly Boit, Seattle, WA (US); Charles E. Murry, Seattle, WA (US); Jennifer Bennett, Seattle, WA (US)

(73) Assignee: University of Washington, Seattle, WA (US)

(21) Appl. No.: 19/106,062

(22) PCT Filed: Aug. 24, 2023
(86) PCT No.: PCT/US2023/072870

§ 371 Date: Feb. 24, 2025

Related U.S. Application Data
(60) Provisional application No. 63/373,530, filed on Aug. 25, 2022.

(51) Int. Cl. A61L 27/52; A61K 38/00; A61L 27/38; A61L 27/54; A61L 27/58; C07K 14/00

(52) U.S. Cl. CPC A61L 27/52; A61L 27/3826; A61L 27/54; A61L 27/58; C07K 14/001; A61K 38/00; A61L 2300/64; A61L 2400/06

(57) ABSTRACT

Embodiments of the present disclosure provide compositions and methods for injectable recombinant protein-based hydrogels for therapeutic delivery. Embodiments of the composition and methods comprise a recombinant protein, a cell consortia, and a biocompatible medium. The recombinant protein comprises one or more self-association domains and a flexible linker comprising a pseudorepeat of amino acids, forming stable hydrogels in contact with the cell consortia. Such composition and methods have broad application in delivery of viable cells to a biological tissue for tissue repair and regeneration.

### CROSS-REFERENCE TO RELATED APPLICATION

This application claims the benefit of U.S. Provisional Patent Application No. 63/373,530, filed Aug. 25, 2022, the contents of which are hereby incorporated by reference in their entirety.

### STATEMENT OF GOVERNMENT LICENSE RIGHTS

This invention was made with government support under Grant Nos. F31HL152626 and R35GM138036, awarded by the National Institutes of Health. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1272WOUW_Seq_List_20230823.xml. The XML file is 78,646 bytes; was created on Aug. 23, 2023; and is being submitted electronically via Patent Center with the filing of the specification.

### BACKGROUND

Direct injection of cells provides a simple and straightforward route to localize therapeutic cells precisely to diseased bodily tissues in a minimally invasive manner. This method has been used to treat debilitating diseases including myocardial infarction, osteoarthritis, and Parkinson's. Although such therapeutic strategies initially seemed promising, engraftment and long-term survival of the injected cells was typically very low (<10%), therefore limiting overall efficacy and imposing substantial barriers in cost and efficiency towards clinical translation. Such poor viability has been attributed to many factors, including cell membrane-damaging shear forces accompanying syringe- and catheter-based injection; a lack of a supportive 3D matrix and its pro-survival signals from cell adhesion; and host inflammatory and immune responses. Methods for robust cell transplantation that address these problem areas remain in great need.

Capable of being delivered through a catheter, insulating against membrane-damaging extensional flow, and supplying a supportive 3D environment in vivo, injectable biomaterials represent an attractive tool towards improving cell retention and subsequent tissue function following transplantation. Such systems are most commonly derived from synthetic polymers [e.g., poly(ethylene glycol), poly(2-hydroxyethyl methacrylate), poly(N-isopropylacrylamide)]. While these synthetic biomaterials afford precise physicochemical tunability, concerns persist over their potential immunogenicity, toxicity, and lack of biodegradability. Alternative injectable materials have been developed from tissue-harvested biomolecules (e.g., collagen, gelatin, alginate, Matrigel®, fibrin, decellularized extracellular matrix). Though generally more supportive of native cell functions, these natural protein-based platforms are limited by a lack of tunability, high polydispersity, and substantial batch-to-batch variability.

Therefore, a need exists for compositions usable in biomedical applications which overcome the foregoing problems, and which additionally exhibit precise user-defined specificity, synthetic scalability through large-scale fermentation, and intrinsic biodegradability.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some aspects, provided herein is an inoculant composition, comprising:
a recombinant protein, a cell consortia, and a biocompatible medium,
wherein the recombinant protein comprises one or more self-association domains, and a flexible linker comprising a pseudorepeat of amino acids, and
wherein the recombinant protein contacts the cell consortia.

In other aspects, provided herein is a method of cell therapy, the method comprising:
forming an inoculant composition, and
injecting the inoculant composition into a biological tissue,
wherein the inoculant composition comprises a recombinant protein, a cell consortia, and a biocompatible medium,
wherein the recombinant protein comprises one or more self-association domains and a flexible linker,
wherein the recombinant protein and cell consortia are disposed in the biocompatible medium, and
wherein the injecting causes shear thinning of the inoculant composition.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A shows RGD cell adhesion sites, coil domains, and XTEN in a coil-flexible linker-coil material design;

FIG. 1B shows monomer units self-associated to form a gel via homopentameric coiled-coil interactions (PDB 1VDF), wherein the PXP pentamers create a gel network, and the physical bonds can be broken under high shear stress and reformed upon return to low shear stress;

FIG. 1C shows amino acid sequences of the original P domain (SEQ ID NO: 1), and amino acid sequences of the single point mutations T40A (SEQ ID NO: 2), Q54A (SEQ ID NO: 3), and T40A+Q54A (SEQ ID NO: 4), which stabilize the coiled-coil interactions;

FIG. 1D shows images of PXP gels±RGD and with mutations to enhance physical association between P domains;

FIG. 1E shows injectability of PXP gels through a 26 gauge (G) needle;

FIG. 2A shows InVision™ His-tag-stained SDS-PAGE of SEQ ID NO:7;

FIG. 2B shows Coomassie-stained SDS-PAGE of SEQ ID NO:7;

FIG. 2C shows mass spectrometry final mass of 29,962.6 Da (calculated mass is 29,948.2 Da) for SEQ ID NO:7;

FIG. 3A shows InVision™ His-tag-stained SDS-PAGE of SEQ ID NO:8;

FIG. 3B shows Coomassie-stained SDS-PAGE of SEQ ID NO:8;

FIG. 3C shows mass spectrometry final mass of 30,907.7 Da (calculated mass is 30,893.8 Da) for SEQ ID NO:8;

FIG. 4A shows InVision™ His-tag-stained SDS-PAGE of SEQ ID NO:9;

FIG. 4B shows Coomassie-stained SDS-PAGE of SEQ ID NO:9;

FIG. 4C shows mass spectrometry final mass of 30,190.4 Da (calculated mass is 30,176.1 Da) for SEQ ID NO:9;

FIG. 5A shows InVision™ His-tag-stained SDS-PAGE of SEQ ID NO:10;

FIG. 5B shows Coomassie-stained SDS-PAGE of SEQ ID NO:10;

FIG. 5C shows mass spectrometry final mass of 30,122.5 Da (calculated mass is 30,122.1 Da) for SEQ ID NO:10;

FIG. 6A shows InVision™ His-tag-stained SDS-PAGE of SEQ ID NO:11;

FIG. 6B shows Coomassie-stained SDS-PAGE of SEQ ID NO:11;

FIG. 6C shows mass spectrometry final mass of 30,062.0 Da (calculated mass is 30,062.0 Da) for SEQ ID NO:11;

FIG. 7A shows rheometric analysis of coiled-coil protein-based hydrogels wherein G′: storage modulus represented by dark-colored closed circles, G″: loss modulus represented by light-colored open circles, and strain sweeps for all gel types at 37° C. and 30 rad s⁻¹ from 0-500% strain; shear-thinning or injectable behavior is observed for all gel types by strain crossover points (G″>G′) indicated by arrows;

FIG. 7B shows rheometric analysis of coiled-coil protein-based hydrogels wherein G′: storage modulus represented by dark-colored closed circles, G″: loss modulus represented by light-colored open circles, and frequency sweeps for all gel types at 37° C. and 5% strain from 0.1-100 rad s⁻¹; Frequency crossover points indicated by arrows; a lengthened linear viscoelastic range (LVER) is observed after the introduction of point mutations as demonstrated by the length of plateau storage modulus (G′) in frequency sweeps;

FIG. 7C shows rheometric analysis of coiled-coil protein-based hydrogels wherein G′: storage modulus represented by dark-colored closed circles, G″: loss modulus represented by light-colored open circles, and strain crossover values reported from strain sweeps showing a statistically significant decrease (p<0.05) in strain crossover with the introduction of both mutations (T40A+Q54A) at 37° C. and 25° C.;

FIG. 7D shows rheometric analysis of coiled-coil protein-based hydrogels wherein G′: storage modulus represented by dark-colored closed circles, G″: loss modulus represented by light-colored open circles, and frequency crossover reported from frequency sweeps indicating improved stability with mutations due to increased LVER (no crossover for Q54A and T40A+Q54A); a multiple comparisons two-way ANOVA table was implemented for mutant comparison (* p<0.05, ** p<0.01, **** p<0.0001);

FIG. 8A shows self-healing properties of coiled-coil protein-based hydrogels wherein G′: storage modulus represented by dark-colored closed circles, G″: loss modulus represented by light-colored open circles, and cyclic strain sweep test at 37° C. and 30 rad s⁻¹ with 5% low strain value and 500% high strain value; full recovery is achieved for each gel type after each of 4 periods of high strain;

FIG. 8B shows self-healing properties of coiled-coil protein-based hydrogels wherein G′: storage modulus represented by dark-colored closed circles, G″: loss modulus represented by light-colored open circles, and zoom of a representative high strain period from the cyclic strain sweep; visually slower recovery of PXP is observed when compared to the mutants FIG. 8C shows self-healing properties of coiled-coil protein-based hydrogels wherein G′: storage modulus represented by dark-colored closed circles, G″: loss modulus represented by light-colored open circles, and average recovery time crossover, when G′>G″ after the high strain period, is improved for mutants by 3 s at 37° C.;

FIG. 8D shows self-healing properties of coiled-coil protein-based hydrogels wherein G′: storage modulus represented by dark-colored closed circles, G″: loss modulus represented by light-colored open circles, and average storage modulus for each gel type (10% w/w) and temperature conditions with no statistical difference between mutants; a multiple comparisons two-way ANOVA table was implemented for mutant comparison (* p<0.05);

FIG. 9A shows biodegradation of 10% (w/w) PXP gels with images at days 0, 3, 6, 12, and 31 of degradation in PBS at 37° C. for each gel type; slowest degradation is observed in the T40A+Q54A mutant; gels are visually intact after 31 days;

FIG. 9B shows biodegradation of 10% (w/w) PXP gels with BCA analysis of % degraded into PBS solution after incubation for 31 days; a plateau degradation is observed after the first 11 days for all gel types;

FIG. 9C shows biodegradation of 10% (w/w) PXP gels with % degraded during the first 15 days; slower degradation is observed with the introduction of point mutations, with the slowest degradation being T40A+Q54A; error bars reported as SEM, N=3;

FIG. 10 shows hESC-CM injection protection; viability of cells before injection (left); viability of cells immediately after injection through a 26 G needle in cell suspension (center); or cells encapsulated in PXP (right), as determined by a NucleoCounter®; 10% improvement upon viability post injection by encapsulating cells within PXP; statistically significant decrease in viability post injection of cell suspension; no significance between pre-injection and cells with PXP; a multiple comparisons one-way ANOVA table was implemented for statistical analysis (**** p<0.0001, ns=not significant);

FIG. 11A shows hESC-CM and fibroblast viability 24-72 hours post injection and confocal images of injected PXP encapsulated fibroblasts and injected fibroblast suspension group 24 hours post injection; Ethidium Homodimer-1 stained for dead cells wherein 70% ethanol treated cells were used as a dead control group;

FIG. 11B shows hESC-CM and fibroblast viability 24-72 hours post injection and confocal images of injected PXP encapsulated fibroblasts and injected fibroblast suspension group 24 hours post injection; calceinAM stained for live cells and Ethidium Homodimer-1 stained for dead cells;

FIG. 11C shows hESC-CM and fibroblast viability 24-72 hours post injection and confocal images of injected PXP encapsulated fibroblasts and injected fibroblast suspension group 24 hours post injection; calceinAM stained for live cells;

FIG. 11D shows hESC-CM and fibroblast viability 24-72 hours post injection with fibroblast viability 24 hrs post injection quantified by live/dead count from confocal images in LAS X software;

FIG. 11E shows hESC-CM and fibroblast viability 24-72 hours post injection with fibroblast viability 72 hrs post injection quantified by a NucleoCounter®;

FIG. 11F shows hESC-CM and fibroblast viability 24-72 hours post injection with hESC-CM viability 72 hrs post injection quantified by a NucleoCounter®;

FIG. 11G shows hESC-CM and fibroblast viability 24-72 hours post injection with hESC-CM viability with a pro survival cocktail (PSC) quantified by NucleoCounter®; one-way ANOVA tables were implemented for statistical analysis (* p<0.05, *** p<0.001, **** p<0.0001, ns=not significant);

FIG. 12A shows strain and frequency sweeps at 25° C. and shear thinning tests at 25° C. and 37° C. for PXP, T40A, Q54A, and T40A+Q54A wherein G′: storage modulus is represented by dark-colored closed circles, G″: loss modulus is represented by light-colored open circles, and representative strain sweep at 25° C. (30 rad s⁻¹, 0-500% strain);

FIG. 12B shows strain and frequency sweeps at 25° C. and shear thinning tests at 25° C. and 37° C. for PXP, T40A, Q54A, and T40A+Q54A wherein G′: storage modulus is represented by dark-colored closed circles, G″: loss modulus is represented by light-colored open circles, and representative frequency sweep at 25° C. (5% strain, 0.1-100 rad s⁻¹);

FIG. 12C shows strain and frequency sweeps at 25° C. and shear thinning tests at 25° C. and 37° C. for PXP, T40A, Q54A, and T40A+Q54A wherein G′: storage modulus is represented by dark-colored closed circles, G″: loss modulus is represented by light-colored open circles, and representative cyclic strain sweep at 25° C. (30 rad s⁻¹, 5% low strain, 500% high strain) demonstrating full recovery of all gels after 4 periods of high strain;

FIG. 12D shows strain and frequency sweeps at 25° C. and shear thinning tests at 25° C. and 37° C. for PXP, T40A, Q54A, and T40A+Q54A wherein G′: storage modulus is represented by dark-colored closed circles, G″: loss modulus is represented by light-colored open circles, and zoom on a representative high strain cycle of the cyclic strain sweep;

FIG. 12E shows strain and frequency sweeps at 25° C. and shear thinning tests at 25° C. and 37° C. for PXP, T40A, Q54A, and T40A+Q54A, and wherein representative rotational shear thinning test at room temperature (25° C.) indicates lowered viscosity at increased shear rates;

FIG. 12F shows strain and frequency sweeps at 25° C. and shear thinning tests at 25° C. and 37° C. for PXP, T40A, Q54A, and T40A+Q54A, and representative rotational shear thinning at physiological temperature (37° C.);

FIG. 13A shows bioconjugation of protein of interest wherein PeGFP contains a single P domain with both mutations (T40A+Q54A) attached to eGFP;

FIG. 13B shows bioconjugation of protein of interest wherein PXP gels are functionalized with the PeGFP protein;

FIG. 13C shows bioconjugation of protein of interest wherein fluorescence of PXP gel with 6.5% coiled-coil interactions functionalized with PeGFP (left) next to an unfunctionalized PXP gel (right) confirms proper refolding after denaturing purification (λ_{excitation}=488 nm, λₑₘᵢₛₛᵢₒₙ=530 nm); and

FIG. 14 shows a photodegradable linker incorporated into the hydrogel, wherein the single-strand, recombinant protein-based hydrogel is crosslinked by self-association domain interactions; exhibits shear-thinning and self-healing for injectability; and is selectively degradable in response to 405 nm light.

### DETAILED DESCRIPTION

The present disclosure describes a therapeutic for biological tissue repair and regeneration.

Biomaterials derived from recombinant proteins afford opportunities for biomedical applications as they exhibit precise user-defined sequence specificity, synthetic scalability through large-scale fermentation, and intrinsic biodegradability. Through inclusion of structural domains that support interprotein physical association, the recombinant protein system described herein can undergo shear-thinning and rapid self-healing that affords direct injectability.

Provided herein is an inoculant composition comprising a recombinant protein, a cell consortia, and a biocompatible medium,
wherein the recombinant protein comprises one or more self-association domains, and a flexible linker comprising a pseudorepeat of amino acids, and
wherein the recombinant protein contacts the cell consortia.

In some embodiments, the flexible linker is intrinsically unstructured.

As used herein, “intrinsically unstructured” means a protein or peptide sequence that lacks ordered secondary or tertiary structure. For example, an intrinsically unstructured protein or peptide sequence lacks an alpha helix, a beta sheet, or a fixed or ordered tertiary structure. The intrinsically unstructured protein can adopt multiple configurations.

In some embodiments, the inoculant composition comprises a pseudorepeat of the amino acids which make up the flexible linker.

As used herein, “pseudorepeat” means a defined number of amino acids which are not the same, and which are repeated in an order which is not regular. For example, a pseudorepeat can be a repeat of 5, a repeat of 6, or a repeat of 7 different amino acids, or amino acids which have a different identity. In a pseudorepeat of amino acids, the total number of amino acids comprises different amino acids (e.g., 5, 6, or 7), which are repeated to make up the total number of amino acids, but wherein the different amino acids are repeated such that no intentional pattern of the repeated amino acids exists. For example, the repeat of a pseudorepeat of 5 different amino acids having the generic identity a, b, c, d, and e does not comprise a pattern such as . . . a-b-c-d-e-a-b-c-d-e-a-b-c-d-e . . . ; . . . a-a-a-b-b-b-c-c-c-d-d-d-e-e-e . . . ; or any other regular type pattern.

The different amino acids in a pseudorepeat are necessarily individually repeated to make up the total number of amino acids of the peptide or protein sequence. For example, a protein comprising 5 different amino acids which make up 100 total amino acids, can be made up of an equal amount of each of the 5 different amino acids, or can be made up of an amount of the 5 different amino acids which is not the same as between any or all of the 5 different amino acids. For example, a protein comprising 100 total amino acids can be comprised of 20 residues of amino acid “a,” 20 residues of amino acid “b,” 20 residues of amino acid “c,” 20 residues of amino acid “d,” and 20 residues of amino acid “e,” when the different amino acids exist in the protein at an identical or equal amount. Alternatively, the different amino acids can exist in the protein in an amount which is not identical or equal. For example, a protein comprising 100 total amino acids can consist of 20 residues of amino acid “a,” 15 residues of amino acid “b,” 35 residues of amino acid “c,” 10 residues of amino acid “d,” and 20 residues of amino acid “e.”

The quantity of the most frequent amino acid can be up to about 5 times the quantity of the least frequent amino acid, up to about 4 times the quantity of the least frequent amino acid, up to about 3 times the quantity of the least frequent amino acid, or up to about 2 times the quantity of the least frequent amino acid.

In some embodiments, the flexible linker consists of a pseudorepeat of 3 different amino acid classes. For example, the flexible linker can comprise one amino acid class wherein the amino acid side chain is charged at physiological pH (e.g., aspartic acid, glutamic acid, lysine, arginine, histidine, or a combination thereof); one amino acid class wherein the amino acid side chain is polar (e.g., serine, threonine, cysteine, tyrosine, asparagine, glutamine, or a combination thereof); and one amino acid class wherein the amino acid side chain is nonpolar (e.g., glycine, alanine, proline, valine, isoleucine, leucine, methionine, tryptophan, phenylalanine, or a combination thereof).

In some embodiments, the flexible linker consists of a pseudorepeat of 5 different amino acids, a pseudorepeat of 6 different amino acids, or a pseudorepeat of 7 different amino acids. In some embodiments, the flexible linker consists of a pseudorepeat of 6 different amino acids.

In some embodiments, the flexible linker consists of a pseudorepeat of 5-7 amino acids, wherein one or two different amino acids have a side chain which is charged at physiological pH (e.g., aspartic acid, glutamic acid, lysine, arginine, histidine, or a combination thereof); one or two different amino acids have a side chain which is polar (e.g., serine, threonine, cysteine, tyrosine, asparagine, glutamine, or a combination thereof); and one, two, three, or four amino acids have a side chain which is nonpolar (e.g., glycine, alanine, proline, valine, isoleucine, leucine, methionine, tryptophan, phenylalanine, or a combination thereof).

In some embodiments, the flexible linker consists of a pseudorepeat of 5-7 different amino acids, wherein one or two different amino acids have a negatively charged side-chain at physiological pH (e.g., aspartic acid, glutamic acid); one or two different amino acids have a polar side chain (e.g., serine, threonine); and one, two, or three, different amino acids have a side chain which is nonpolar (e.g., glycine, alanine, proline).

In some embodiments, the flexible linker consists of a pseudorepeat of 6 different amino acids, wherein one amino acid has a negatively charged side-chain at physiological pH (e.g., aspartic acid or glutamic acid); two different amino acids have a polar side chain (e.g., serine, threonine); and three different amino acids have a nonpolar side chain (e.g., glycine, alanine, proline).

In some embodiments, the flexible linker consists of amino acids alanine, glutamic acid, glycine, proline, serine, and threonine.

In some embodiments, the flexible linker is an XTEN linker. The flexible linker, as used herein, can be denoted “X.”

In some embodiments, the flexible linker consists of between 36-3,456 amino acids, between 36-1,728 amino acids, between 36-864 amino acids, between 36-600 amino acids, between 36-400 amino acids, between 36-200 amino acids, between 100-400 amino acids, between 100-300 amino acids, between 100-200 amino acids, between 120-160 amino acids, between 130-150 amino acids, 142 amino acids, at least 100 amino acids, or up to 200 amino acids. In some embodiments, the flexible linker consists of 142 amino acids. In some embodiments, the flexible linker consists of more than 864 amino acids. In some embodiments, the flexible linker consists of 900-3,456 amino acids.

In some embodiments, the flexible linker has the sequence PAGSPTSTEEGTSESATPESGPGTSTEPSEGSAPGSPAGSPTSTEEGTSTEPSEGSAP GTSTEPSEGSAPGTSESATPESGPGSEPATSGSETPGSEPATSGSETPGSPAGSPTST EEGTSESATPESGPGTSTEPSEGSAP (SEQ ID NO: 5).

In some embodiments, the recombinant protein comprises one or more self-association domains. In some embodiments, the recombinant protein comprises two self-association domains. In embodiments, wherein the recombinant protein comprises more than one self-association domains, or comprises two self-association domains, the recombinant protein comprises a first self-association domain and a second self-association domain.

In some embodiments comprising a first self-association domain and a second self-association domain, the first self-association domain has a sequence that is the same as the second self-association domain. In some embodiments comprising a first self-association domain and a second self-association domain, the first self-association domain has a sequence that is different from the second self-association domain.

In some embodiments, the first self-association domain consists of any one of SEQ ID NOs: 1-4, and the second self-association domain consists of any one of SEQ ID NOs: 1-4.

In some embodiments, each self-association domain is structured, and the structure comprises a coil or an alpha-helix. As used herein, a coil is the secondary structure of a sequence of amino acids arranged to form an alpha helix, as known to one having ordinary skill in the art. Specifically, a coil is a twisted configuration of the amino acid sequence wherein each turn of the coil comprises about 3.6 amino acid residues.

In some embodiments, the first self-association domain comprises a coil, and the second self-association domain comprises a coil.

In some embodiments, the recombinant protein comprises a cell adhesion motif at a first terminus of the recombinant protein, a cell adhesion motif at a second terminus of the recombinant protein, or a cell adhesion motif at both a first and a second terminus of the recombinant protein.

In some embodiments, the cell adhesion motif of the first terminus and the cell adhesion motif of the second terminus are the same. In some embodiments, the cell adhesion motif of the first terminus and the cell adhesion motif of the second terminus are different.

In some embodiments, the cell adhesion motif is covalently bound to the self-association domain.

In some embodiments, the cell adhesion motif binds to a cell.

In some embodiments, the cell adhesion motif has a sequence selected from the group consisting of GRGDS (SEQ ID NO: 6); PHSRN (SEQ ID NO: 7); DELPQLPHPNLHGPEILDVPS (SEQ ID NO: 8); REDV (SEQ ID NO: 9); YEKPGSPPREVVPRPRPGV (SEQ ID NO: 10); KNNQKSEPLIGRKKT (SEQ ID NO: 11); YRYRYTPKEKTGPMKE (SEQ ID NO: 12); SPPRRARVT (SEQ ID NO: 13); WQPPRARI (SEQ ID NO: 14); KLDAPT (SEQ ID NO: 15); TDIDAPS (SEQ ID NO: 16); SRARKNAASIKVAVSADR (SEQ ID NO: 17); RGDN (SEQ ID NO: 18); KATPMLKMRTSFHGCIK (SEQ ID NO: 19); KEGYKVRLDLNITLEFRTTSK (SEQ ID NO: 20); KNLEISRSTFDLLRNSYGVRK (SEQ ID NO: 21); DGKWHTVKTEYIKRKAF (SEQ ID NO: 22); KQNCLSSRASFRGCVRNLRLSR (SEQ ID NO: 23); NRWHSIYITRFG (SEQ ID NO: 24); TWYKIAFQRNRK (SEQ ID NO: 25); RKRLQVQLSIRT (SEQ ID NO: 26); YIGSRC (SEQ ID NO: 27); PDSGR (SEQ ID NO: 28); RYVVLPRPVCFEKGKGMNYVR (SEQ ID NO: 29); LGTIPG (SEQ ID NO: 30); YGYYGDALR (SEQ ID NO: 31); AFGVLALWGTRV (SEQ ID NO: 32); DSITKYFQMSLE (SEQ ID NO: 33); VILQQSAADIAR (SEQ ID NO: 34); RNIAEIIKDI (SEQ ID NO: 35); KAFDITYVRLKF (SEQ ID NO: 36); TDIRVTLNRLNTF (SEQ ID NO: 37); SETTVKYIFRLHE (SEQ ID NO: 38); TSIKIRGTYSER (SEQ ID NO: 39); RGDS (SEQ ID NO: 40); RGDF (SEQ ID NO: 41); HHLGGAKQAGDV (SEQ ID NO: 42); KRLDGGS (SEQ ID NO: 43); CQEPGGLVVPPTDAP (SEQ ID NO: 44); LCDLAPEAPPPTLPP (SEQ ID NO: 45); DLVFLLDGSSRLSEAEFEVLKAFVVDE (SEQ ID NO: 46); RGDV (SEQ ID NO: 47); SIGFRGDTC (SEQ ID NO: 48); VTCG (SEQ ID NO: 49); CSVTCG (SEQ ID NO: 50); VDAVRTEKGFLLLASLRQMKKTRGTLLALERKDHS (SEQ ID NO: 51); FQGVLQNVRFVF (SEQ ID NO: 52); RFYVVMWK (SEQ ID NO: 53); RGDTP (SEQ ID NO: 54); SRGDTG (SEQ ID NO: 55); DGEA (SEQ ID NO: 56); GVKGDKGNPGWPGAP (SEQ ID NO: 57); TAGSCLRKFSTM (SEQ ID NO: 58); GEFYFDLRLKGDK (SEQ ID NO: 59); CNYYSNSYSFWLASLNPER (SEQ ID NO: 60); FYFDLR (SEQ ID NO: 61); FTLCPR (SEQ ID NO: 62); RHDS (SEQ ID NO: 63); EPRGDNYR (cyclic) (SEQ ID NO: 64); PSITWRGDGRDLQEL (SEQ ID NO: 65); VLYGPRLDERDAPGNWTWPENSQQTPMC (SEQ ID NO: 66); KELLLPGNNRKV (SEQ ID NO: 67); PSKVILPRGGC (cyclic) (SEQ ID NO: 68); CPCFLLGCC (cyclic) (SEQ ID NO: 69); GKSFTIECRVPTVEP (SEQ ID NO: 70); KYSFNYDGSE (SEQ ID NO: 71); QIDSP (cyclic) (SEQ ID NO: 72); IDSP (SEQ ID NO: 73); GNEH (SEQ ID NO: 74); KLEK (SEQ ID NO: 75); LRAHAVDVNG (SEQ ID NO: 76); YSDNGTF (SEQ ID NO: 77); PPRWGLRNRPIN (SEQ ID NO: 78); and GRYDS (SEQ ID NO: 79).

In some embodiments, the cell adhesion motif has the sequence GRGDS (SEQ ID NO: 6). In some embodiments, the cell adhesion motif has the sequence PHSRN (SEQ ID NO: 7).

In some embodiments, the recombinant protein comprises a single strand. As used herein, a “single strand” means the recombinant protein is one contiguous strand of amino acids covalently bound together. In some embodiments, the recombinant protein consists of one or more self-association domain, and each of the one or more self-association domains is covalently bound to the flexible linker. In some embodiments, the recombinant protein consists of one or more cell adhesion motif covalently bound to one or more self-association domain, and each of the one or more self-association domains is covalently bound to the flexible linker. For example, the single strand of the recombinant protein consists of any one of SEQ ID NOs: 80-84.

In some embodiments, the first self-association domain and the second self-association domain of the recombinant protein self-associate and interact intramolecularly through non-covalent bonds. In some embodiments, the first self-association domain forms a reversible intramolecular interaction with the second self-association domain of the recombinant protein.

In some embodiments, the recombinant protein interacts with one to five additional recombinant proteins. In some embodiments, the recombinant protein interacts with four additional recombinant proteins to form a pentameric unit, or a homopentameric structure. In such embodiments, each recombinant protein interacts non-covalently with the additional one to five, or four, recombinant proteins. The one or more self-association domain of each recombinant protein interacts non-covalently with one or more self-association domain of another recombinant protein.

In some embodiments, interaction of the recombinant protein with one to five additional recombinant proteins results in a hydrogel formation. In some embodiments, interaction of the recombinant protein with four additional recombinant proteins results in a hydrogel formation.

In some embodiments, the first self-association domain of the recombinant protein forms a reversible intermolecular interaction with the first self-association domain, the second self-association domain, or a combination thereof, of at least one of the one to five additional recombinant proteins. In some embodiments, the second self-association domain of the recombinant protein forms a reversible intermolecular interaction with the first self-association domain, the second self-association domain, or a combination thereof, of at least one of the one to five additional recombinant proteins.

In some embodiments, the amino acid sequence of the first self-association domain of the recombinant protein is the same as the amino acid sequence of the first self-association domain, the second self-association domain, or both the first and second self-association domains, of each of the one to five additional recombinant proteins.

In some embodiments, the amino acid sequence of the second self-association domain of the recombinant protein is the same as the amino acid sequence of the first self-association domain, the second self-association domain, or both the first and second self-association domains, of each of the one to five additional recombinant proteins.

In some embodiments, the amino acid sequence of the first self-association domain of the recombinant protein is different from the amino acid sequence of the first self-association domain, the second self-association domain, or both the first and second self-association domains, of each of the one to five additional recombinant proteins.

In some embodiments, the amino acid sequence of the second self-association domain of the recombinant protein is different from the amino acid sequence of the first self-association domain, the second self-association domain, or both the first and second self-association domains, of each of the one to five additional recombinant proteins.

In some embodiments, the amino acid sequence of the first self-association domain of the recombinant protein is the same as the amino acid sequence of the first self-association domain, the second self-association domain, or both the first and second self-association domains, of each of the four additional recombinant proteins.

In some embodiments, the amino acid sequence of the second self-association domain of the recombinant protein is the same as the amino acid sequence of the first self-association domain, the second self-association domain, or both the first and second self-association domains, of each of the four additional recombinant proteins.

In some embodiments, the amino acid sequence of the first self-association domain of the recombinant protein is different from the amino acid sequence of the first self-association domain, the second self-association domain, or both the first and second self-association domains, of each of the four additional recombinant proteins.

In some embodiments, the amino acid sequence of the second self-association domain of the recombinant protein is different from the amino acid sequence of the first self-association domain, the second self-association domain, or both the first and second self-association domains, of each of the four additional recombinant proteins.

In some embodiments, the inoculant composition comprises a photodegradable linker. As used herein, a “photodegradable linker” is a molecule which links portions of the recombinant protein. In some embodiments, the photodegradable linker is selected from the group consisting of PhoCl1, PhoCl2c, and PhoCl2f. In some embodiments, the photodegradable linker is connected to one or more amino acids of the flexible linker. In some embodiments, the photodegradable linker is connected to any two amino acids of the flexible linker. In some embodiments, the photodegradable linker is connected to any two amino acids of the flexible linker at a position within about 40 percent, about 30 percent, about 20 percent, or about 10 percent of the mid-point of the flexible linker amino acid sequence. In some embodiments, the photodegradable linker is connected to a self-association domain. The photodegradable linker is degraded upon exposure to a single wavelength or a range of wavelengths of the electromagnetic spectrum. In some embodiments, the photodegradable linker is degraded at a wavelength between about 365 nm and about 420 nm.

In some embodiments, the cell consortia comprises the same types of cells. In some embodiments, the cell consortia comprises different types of cells. In some embodiments, the cells are fibroblasts, cardiomyocytes, stromal cells, neural cells, islet cells, embryonic stem cells, induced pluripotent stem cells, mesenchymal stem cells, neural cells, retinal cells, or a combination thereof.

In some embodiments, the weight of the cell consortia to the weight of the recombinant protein is between about 2% and about 20%, between about 2% and about 15%, between about 2% and about 12%, between about 2% and about 10%, between about 3% and about 8%, between about 4% and about 6%, is no more than about 20%, is no more than about 12%, is no more than about 8%, is at least 2%, is about 2.5%, or is about 5%.

In some embodiments, the recombinant protein is connected to a cell of the cell consortia through the cell adhesion motif.

In some embodiments, the recombinant protein is connected to a cell of the cell consortia by interacting with one or more antigen, one or more growth factor, one or more cell binding motif, one or more cytokine, one or more immunomodulator, or a combination thereof, of the cell.

In some embodiments, the cell consortia is in contact with the hydrogel. In some embodiments, the cell consortia is encapsulated by the hydrogel.

In some embodiments, the recombinant protein and cell consortia are disposed in the biocompatible medium.

In some embodiments, the biocompatible medium comprises water, one or more nutrients, one or more biocompatible constituents, or a combination thereof.

In some embodiments, the one or more nutrients comprises a buffer, salt, BcL-XL BH4, cyclosporin A, pinacidil, IGF-1, ZVAD, RPMI, DMEM, fetal bovine serum, bovine serum albumin, or a combination thereof.

In some embodiments, the one or more biocompatible constituents comprises collagen, silk, resillin, gelatin, alginate, Matrigel, decellularized extracellular matrix, fibrin, PEG, zwitterionic polymer, PHEMA, PNIPAAM, hyaluronic acid, or a combination thereof.

In some embodiments, a cell of the inoculant composition is fused to a cell of a biological tissue, wherein the cell of the biological tissue is a cell from a heart, a muscle, an eye, a brain, a pancreas, or a skin. In some embodiments, a cell of the inoculant composition is fused to a cell of a biological tissue through one or more than one cell binding motif.

In another aspect, the present disclosure provides a method of cell therapy, the method comprising:
forming an inoculant composition as described herein, and
injecting the inoculant composition into a biological tissue,
wherein the inoculant composition comprises a recombinant protein, a cell consortia, and a biocompatible medium, each as described herein,
wherein the recombinant protein comprises one or more self-association domains and a flexible linker, each as described herein,
wherein the recombinant protein and cell consortia are disposed in the biocompatible medium, and
wherein the injecting causes shear thinning of the inoculant composition.

In some embodiments, the intramolecular interactions, intermolecular interactions, or a combination thereof, of the self-association domains are reversibly broken upon the injecting.

In some embodiments, the injecting causes shear thinning upon a flow of the inoculant composition through a canal having a diameter of about 16 to about 30 gauge, of about 20 to about 30 gauge, of about 22 to about 28 gauge, of at least 20 gauge, of no more than 30 gauge, or of about 26 gauge.

In some embodiments, the shear thinning is caused by the combination of the canal diameter with the flow rate of the inoculant composition through the canal.

In some embodiments, the injecting the inoculant composition results in an increased cell count in the biological tissue. Specifically, after injecting the inoculant composition, the biological tissue has more cells than before the inoculant composition was injected, at a time immediately after injection, about 1 day after injection, about 2 days after injection, about 3 days after injection, about 4 days after injection, about 5 days after injection, about 6 days after injection, about 7 days after injection, about 10 days after injection, about 2 weeks after injection, about 3 weeks after injection, about 4 weeks after injection, about 6 weeks after injection, about 12 weeks after injection, at least about 3 days after injection, or at least about 2 weeks after injection.

In some embodiments, the viability of the cells in the biological tissue after the injection is greater than the viability of the cells in the biological tissue before the injection, by about 50% to about 90%, by about 60% to about 85%, by about 65% to about 85%, by about 70% to about 80%, by about 72% to about 78%, by at least about 65%, by up to about 85%, or by about 75%.

In some embodiments, the biological tissue comprises a cell from a heart, a muscle, an eye, a brain, a pancreas, or a skin.

In some embodiments, the biological tissue is tissue from a mammal. In some embodiments, the biological tissue is tissue from a human. In some embodiments, the cells of the cell consortia are cells from a mammal. In some embodiments, the cells of the cell consortia are cells from a human.

In another aspect, the disclosure comprises a kit, wherein the kit comprises the inoculant composition as described herein, a canal by which the inoculant composition is injected into the biological tissue, a device or mode for causing the injecting, instructions, or a combination thereof.

The term “about” refers to an acceptable degree of error for the quantity measured given the nature or precision of the measurements. Typical, exemplary degrees of error are within ±10% of a given value or range of values. For example, a reference to “about X” specifically indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to teach and provide written support for a claim limitation of, e.g., “0.98X.” Numerical quantities given herein are approximate unless stated otherwise, meaning that the term “about” can be inferred when not expressly stated.

The group “A or B” is equivalent to the group “selected from the group consisting of A and B.”

The terms “a,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells.

### EXAMPLES

### Example 1

### General Methods

Chemical reagents and solvents were purchased and used as received unless otherwise noted. Deionized water (dH₂O) was generated by a U.S. Filter Corporation Reverse Osmosis System with a Desal membrane. Lyophilization was performed on a LABCONCO® FreeZone® 2.5 Plus freeze-dryer equipped with a LABCONCO® rotary vane 117 vacuum pump. Matrix-assisted laser desorption/ionization time of flight (MALDI-TOF) mass spectrometry was performed in reflectron positive ion mode or reflectron negative ion mode on a Bruker® AutoFlex® II using a matrix of α-cyano-4-hydroxycinnamic acid:2,5-dihydroxy benzoic acid (2:1). Whole-protein mass spectrometry was performed using a Waters Synapt®-G2 QTOF. Confocal microscopy was performed on a Leica® Stellaris® 5. Polymerase chain reaction (PCR) was performed in a Bioer LifeECO thermal cycler. Protein expression was performed in a Thermo Scientific® MaxQ 4000 shaker incubator. Cells were lysed using a Fisher Scientific® Model 505 Sonic Dismembrator with a 1.27 cm diameter probe. UV-Vis assays were performed on a BioTek® Synergy HIM plate reader. Fluorescent and true color gel imaging was performed on an Azure 600 AZI600 scanner. Rheological measurements were performed on an Anton Paar Physica MCR301 equipped with a C-PTD200 Peltier plate and a parallel plate geometry (8 mm diameter). Mammalian cell culture was performed in a NuAire® LabGard® ES NU-437 Class II Type A2 Biosafety Cabinet. Cells were maintained in a Sanyo inCu saFe® MCO-17AC incubator at 37° C. and 5% CO₂.

### Example 2

### General Coiled-Coil XTEN-Based Hydrogel

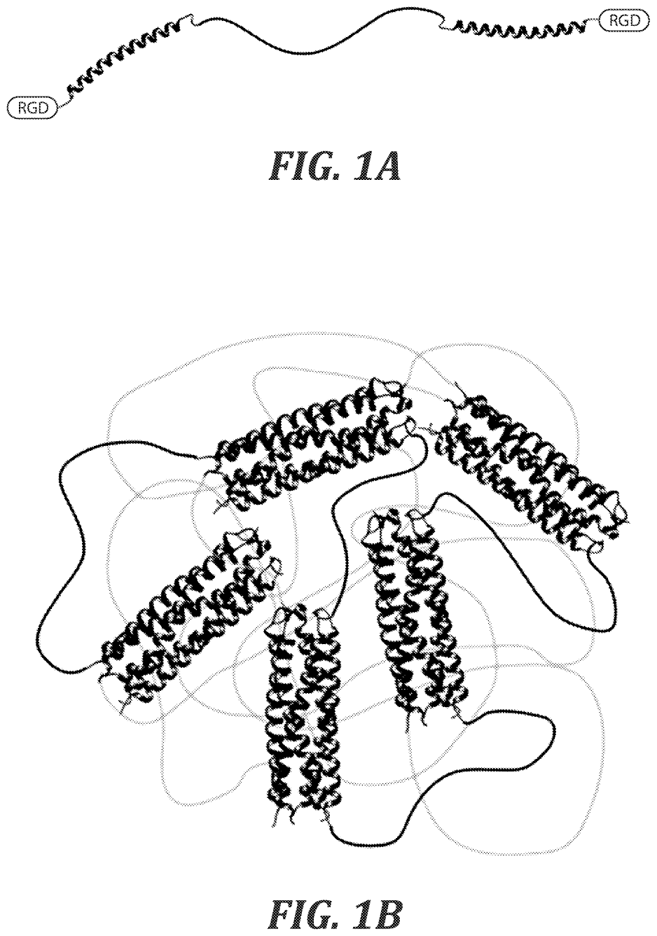

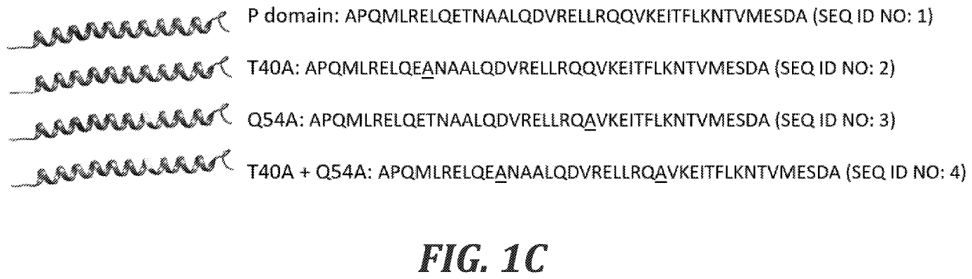

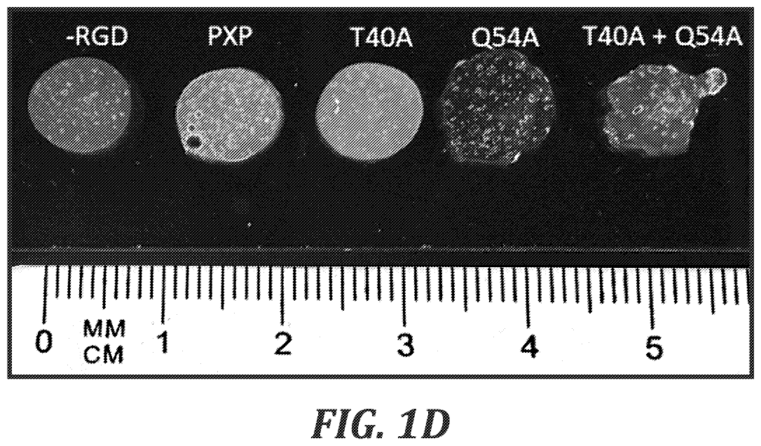

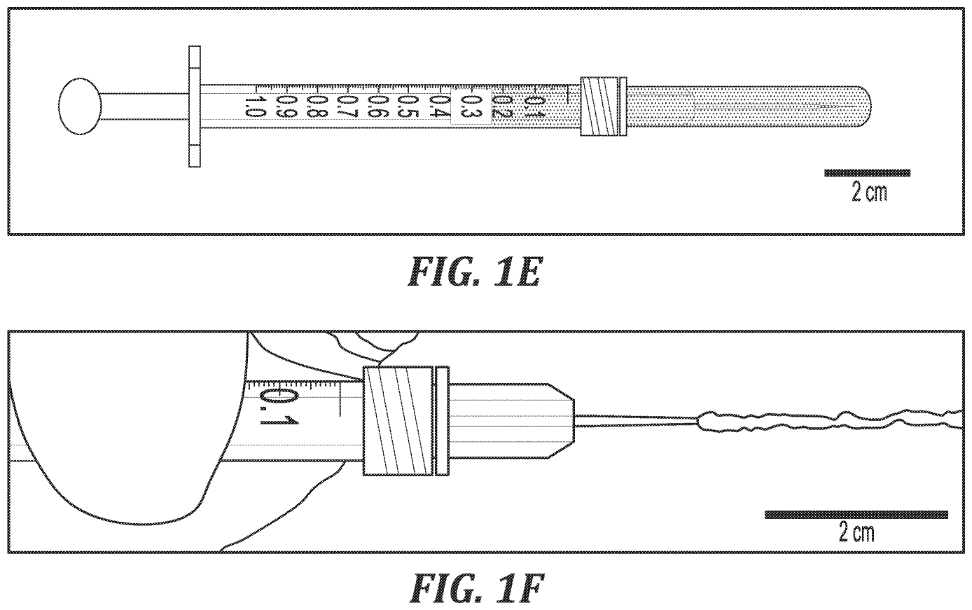

A biomaterial for cell-therapies should be non-immunogenetic, biodegradable, and injectable, display tissue-like elasticity, and support living cells. The design of this disclosure is a synthetic protein biomaterial consisting of a flexible linker flanked by self-association coil domains. The self-association coil domains are derived from the N-terminal fragment of rat cartilage oligomeric matrix protein (COMP), and the flexible linker is XTEN, an unstructured protein which mimics polyethylene glycol (PEG) (FIG. 1A). Cell function could be improved through increased cell-material interactions, so RGD was used, a peptide sequence responsible for cell adhesion found in fibronectin and other ECM proteins. The final construct was designed to include this RGD motif on the terminal ends of the self-association coil domains, to improve cell adhesion.

In the context of this disclosure, the self-association coil comprising peptide sequences, protein domains, proteins, protein fragments, or a combination thereof, are referred to as self-association domains. Examples of self-association domains include the P domain, the T40A mutant, the Q54A mutant, and the T40A+Q54A mutant.

As shown in FIG. 1C, the P domain (SEQ ID NO: 1), mutant T40A (SEQ ID NO: 2), mutant Q54A (SEQ ID NO: 3), and mutant T40A+Q54A (SEQ ID NO: 4) serve as the self-association domain.

The flexible linker XTEN is an engineered protein originally designed to extend the half-life of small molecule drugs as an alternative to PEG. PEG can be attached to therapeutic proteins to increase half-life. However, PEG presents complications of heterogeneity in manufacturing, it is shown to cause renal tubular vacuolation, and there is growing evidence of the presence of anti-PEG antibodies from its overuse. Consequently, alternative options to PEG are needed. One alternative is XTEN, as a genetically encoded strategy which results in consistent manufacturing and lowered immune response (no detectable antibody response). XTEN is comprised of 36 amino acid residue pseudo repeats comprising the amino acids alanine (A), glutamic acid (E), glycine (G), proline (P), serine (S), and threonine (T). The full length of XTEN is 864 amino acids, and the protein conjugate circulation half-life of XTEN can be adjusted by removing or adding pseudo repeats. XTEN's sequence was specifically designed to preclude hydrophobic amino acids phenylalanine (F), isoleucine (I), leucine (L), methionine (M), valine (V), tryptophan (W), and tyrosine (Y), such as to yield a high-expressing nonimmunogenic protein with a flexible structure. XTEN was also designed to exclude other amino acids to improve long-term stability, such as asparagine (N) and glutamine (Q), and to avoid side-chains that bind to cell membranes, such as histidine (H), lysine (K), and arginine (R).

The self-association coil domains, as disclosed herein, self-associate to form a homopentameric structure, or a coiled-coil interaction, as shown in FIG. 1B. By creating triblock ABA-type protein copolymers comprising self-association coil domains, and permitting self-association, the homopentameric structures result. The homopentameric structures are physically stabilized through the reversible association of the self-association coil domains through interactions such as hydrogen bonding, hydrophilic interactions, and van der Waals forces. Homopentameric structure formation results in a hydrogel, which can be injectable.

The strength of these physical stabilization interactions among the self-association coil domains can be modulated by single amino acid mutations within the primary coil amino acid sequence. The single amino acid mutations of this disclosure are shown in FIG. 1C, comprising SEQ ID NOs: 2-4. Mutation of the two polar residues threonine (T) and glutamine (Q) to the sterically smaller and non-polar alanine (A) stabilized the homopentameric helical structure by reducing steric interference between the amino acid side chains. Mutation of the COMP residue 40 from threonine to alanine yielded the mutant protein T40A to produce SEQ ID NO: 2. Similarly, mutation of COMP residue 54 from glutamine to alanine, yielded the mutant protein Q54A to produce SEQ ID NO: 3. Additionally, the self-association protein comprising mutation of both the COMP position 40 T and position 54 Q to A yielded the protein mutant T40A+Q54A to produce SEQ ID NO: 4. The self-association domains lacking either of the T40A or Q54A mutations comprises a sequence identical to the sequence of COMP in many species, including mouse, human, and rat, with the exception of two cysteine residues which were mutated to seine to prevent disulfide bond formation and covalent chemical cross-linking.

### Example 3

### Plasmid Construction

The original plasmid encoding for the construct self-association domain-flexible linker-self-association domain (PXP) comprised sequences coding for self-association domains flanking a truncated XTEN linker (X) and lacking RGD. PXP was designed to comprise a truncated version of XTEN having 144 residues, be flanked by matching self-association domains, and include a 6×His tag (SEQ ID NO: 85). This PXP plasmid lacking RGD additionally comprised a pQE-30 backbone having a T5 promoter, and ampicillin and chloramphenicol acetyltransferase resistance. The resulting protein consists of SEQ ID NO: 80.

The DNA coding for cell adhesion sites RGD, comprising amino acid sequence GRGDS (SEQ ID NO: 6), was cloned into regions between the self-association domains and 6×His tags (SEQ ID NO: 85) on both terminal ends of the self-association domains, resulting in the construct denoted “PXP.” The protein resulting from such plasmid consists of SEQ ID NO: 81. Insertion of RGD at the N terminus was implemented through annealed oligo cloning with BamHI/SalI restriction sites. At the C terminus, Gibson Assembly of a DNA sequence encoding GRGDS (SEQ ID NO: 6) Gblock with overhangs was inserted at the BamHI/SalI restriction sites.

To introduce point mutations in the self-association domains, plasmids encoding for self-association domains in a pTwist Amp High Copy cloning vector (with ampicillin resistance) was used, and a plasmid encoding for X was used, comprising a pQE-30 backbone. Point mutations (T40A, Q54A, or both T40A+Q54A) were introduced by site-directed mutagenesis on P domain plasmids. Mutated self-association P domain plasmids were digested and inserted into the X plasmid at N terminal restriction sites BamHI/SalI and C terminal restriction sites XhoI/HindIII. The protein resulting from such plasmids consists of SEQ ID NOS: 82-84.

To model biofunctionalization, the plasmid encoding for PeGFP (enhanced green fluorescent protein with a single self-association P domain and 6×His tag (SEQ ID NO: 85)) was used with a pET-21b(+) backbone (having a T7 promoter and ampicillin resistance). The protein resulting from such plasmid consists of SEQ ID NO: 86. Deletion of the self-association P domain in the PeGFP plasmid was executed via Gibson assembly to form a sequence encoding eGFP having a 6×His tag (SEQ ID NO: 85) as a control that would not integrate with the homopentameric bundles of the hydrogel network. The protein resulting from such plasmid consists of SEQ ID NO: 87. All cloning sequences were confirmed by Sanger Sequencing.

The amino acid sequences for PXP lacking RGD (SEQ ID NO: 80), PXP (SEQ ID NO: 81), the T40A mutant (SEQ ID NO: 82), the Q54A mutant (SEQ ID NO: 83), the T40A+Q54A mutant (SEQ ID NO: 84), PeGFP (SEQ ID NO: 86), and eGFP (SEQ ID NO: 87) are shown in Table 1 below.

TABLE 1
Amino Acid Sequences.

| Protein | Sequence |
|---|---|
| PXP (-RGD) (SEQ ID NO: 80) | 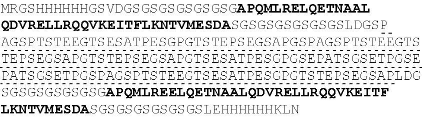 |
| PXP (SEQ ID NO: 81) | 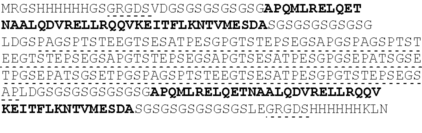 |
| T40A (SEQ ID NO: 82) | 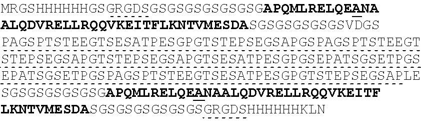 |
| Q54A (SEQ ID NO: 83) | 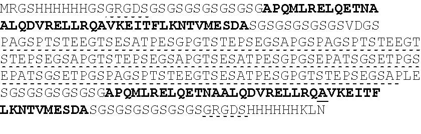 |
| T40A + Q54A (SEQ ID NO: 84) | 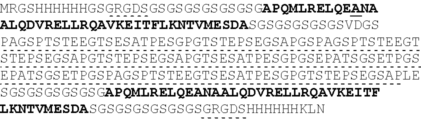 |
| PeGFP | MRGSHHHHHHGSGSGSGSGSGSGSGAPQMLRELQEANAALQD |
| (SEQ ID NO: 86) | VRELLRQAVKEITFLKNTVMESDASGSGSGSGSGSVDMVSKGE |
| | ELFTGVVPILVELDGDVNGHKFSVSGEGEGDATYGKLTLKFICTT |
| | GKLPVPWPTLVTTLTYGVQCFSRYPDHMKQHDFFKSAMPEGYVQ |
| | ERTIFFKDDGNYKTRAEVKFEGDTLVNRIELKGIDFKEDGNILGHK |
| | LEYNYNSHNVYIMADKQKNGIKVNFKIRHNIEDGSVQLADHYQQ |
| | NTPIGDGPVLLPDNHYLSTQSALSKDPNEKRDHMVLLEFVTAAGI |
| | TLGMDELYKKL |
| eGFP | MRGSHHHHHHGSGSGSGSGSGSVDMVSKGEELFTGVVPILVELD |
| (SEQ ID NO: 87) | GDVNGHKFSVSGEGEGDATYGKLTLKFICTTGKLPVPWPTLVTTL |
| | TYGVQCFSRYPDHMKQHDFFKSAMPEGYVQERTIFFKDDGNYKT |
| | RAEVKFEGDTLVNRIELKGIDFKEDGNILGHKLEYNYNSHNVYIM |
| | ADKQKNGIKVNFKIRHNIEDGSVQLADHYQQNTPIGDGPVLLPDN |
| | HYLSTQSALSKDPNEKRDHMVLLEFVTAAGITLGMDELYKKL |

XTEN is shown with dashed underlining, the self-association domains are
shown in bold, RGD is shown with dotted underlining, and the mutations
are the underlined amino acids of the self-association domains.

### Example 4

### Protein Expression, Purification, and Characterization

The plasmids of Example 3 were transformed into BL21(DE3) E. coli cells and protein was expressed in autoinducing media [42.3 mM Na₂HPO₄, 22.04 mM KH₂PO₄, 0.28 M tryptone, 18.23 mM yeast extract, 85.56 mM NaCl, 2.78 mM glucose, 5.84 mM lactose, 0.6% (v/v) glycerol, pH 7.2] supplemented with 0.1 mg mL⁻¹ carbenicillin at 37° C. for 6-8 hours, followed by 18° C. for 14-16 hours. Cell cultures were centrifuged and cell pellets were stored at −80° C. until purification.

Cell pellets were resuspended in equilibration buffer and lysed by sonication on ice (30% amplitude and 33% duty cycle). Clarified lysate was then purified by Ni-NTA affinity chromatography at room temperature. To remove endotoxins, the column was washed 5× with 5 column volumes (CV) of 0.1% Triton X-114 in equilibration buffer followed by 5× with 5 CV of equilibration buffer. Finally, the protein was eluted 4x with 2 CV of elution buffer (4×).

PXP and mutants (T40A, Q54A, and T40A+Q54A) were purified under standard conditions (equilibration buffer: 20 mM Tris, 50 mM NaCl, 10 mM imidazole, pH 8.0; wash buffer: 20 mM Tris, 50 mM NaCl, 15 mM imidazole, 0.1 pH 8.0; elution buffer: 20 mM Tris, 50 mM NaCl, 250 mM imidazole, pH 8.0). The purified protein was dialyzed against deionized water, sterile filtered, flash frozen with liquid nitrogen, and lyophilized to yield a white solid corresponding to the final product.

PeGFP and eGFP proteins were purified under denaturing conditions (equilibration buffer: 0.1 M NaH₂PO₄, 10 mM Tris, 8 M urea, 10 mM imidazole, pH 8.0; elution buffer: 0.1 M NaH₂PO₄, 10 mM Tris, 8 M urea, 250 mM imidazole, pH 4.44). Purified proteins were refolded by a step-down in urea concentration during dialysis. Initially, the protein was dialyzed against 6 M urea in phosphate buffer (13 mM NaH₂PO₄, 86 mM Na₂HPO₄, pH 8.0), followed by 4 M urea in phosphate buffer, then 2 M urea in phosphate buffer, and finally against phosphate buffer without urea. The purified protein solution without urea was flash frozen with liquid nitrogen and stored at −80° C.

Protein purity was assessed using sodium dodecyl sulphate-polyacrylamide gel electrophoresis (SDS-PAGE). Samples were diluted with 2× Laemmli sample buffer containing 2-mercaptoethanol as a reducing agent, and boiled at 100° C. for 10 minutes (min) prior to loading on the gel. SDS-PAGE was run in tris-glycine running buffer at 130 V and stained with InVision™ His Tag stain followed by Coomassie stain. Using the QTRP 5600 Triple-Quad time of flight mass spectrometer, the molecular weight of each protein was confirmed, indicating successful expression of the plasmid of interest.

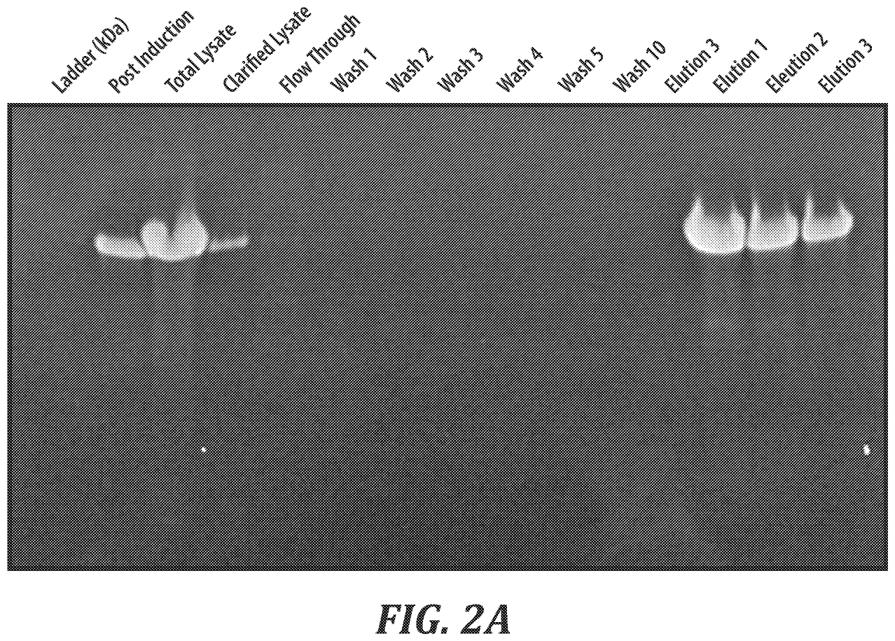

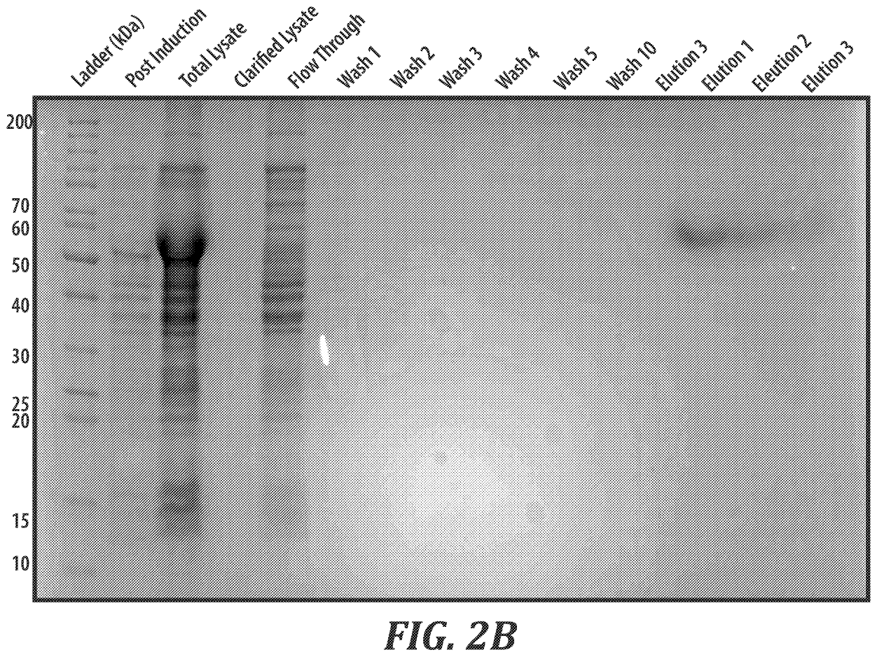

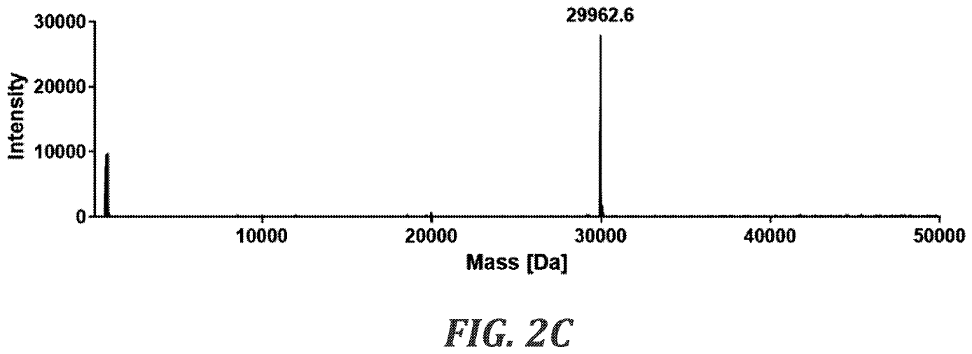

Protein verification of PXP without RGD (SEQ ID NO:80) is shown in FIGS. 2A-2C.

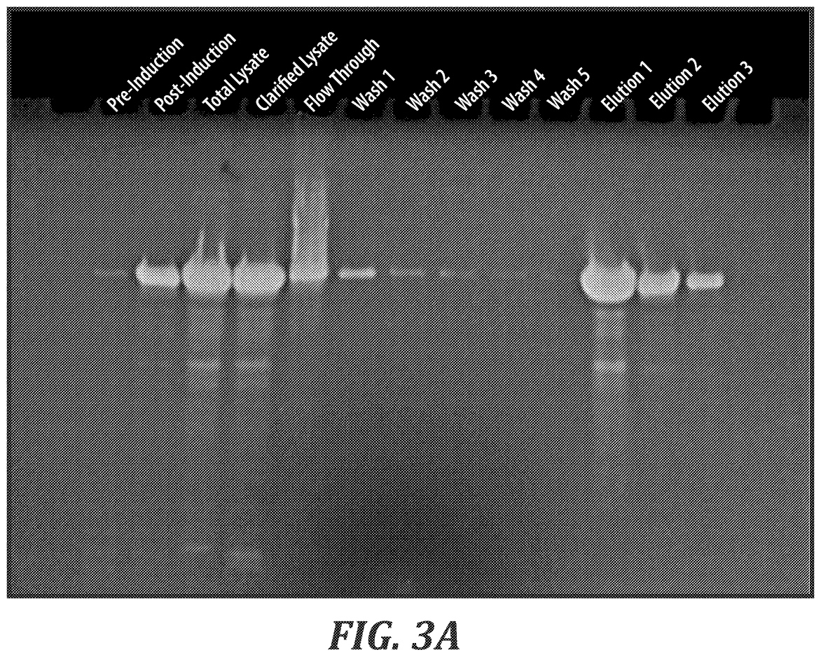

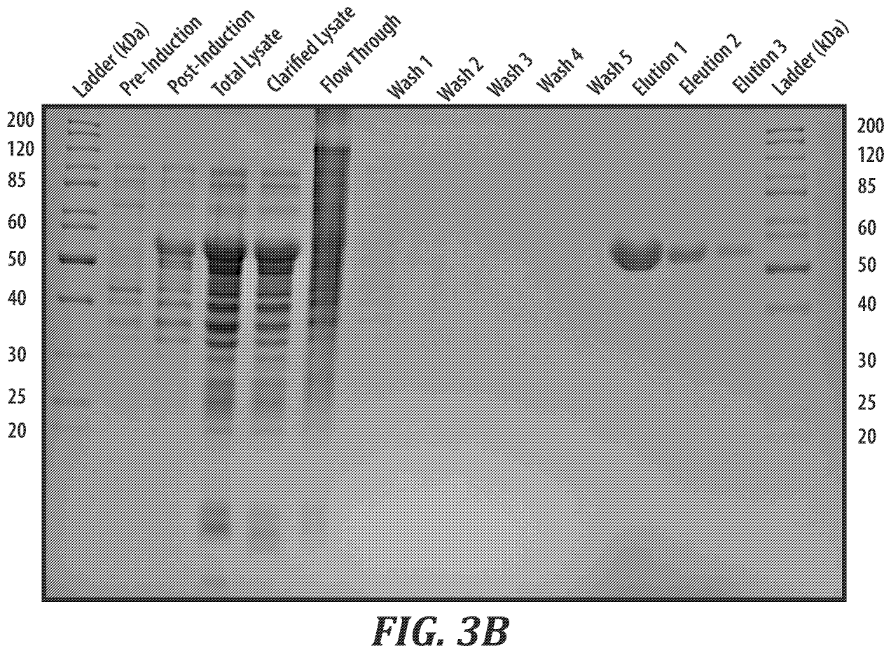

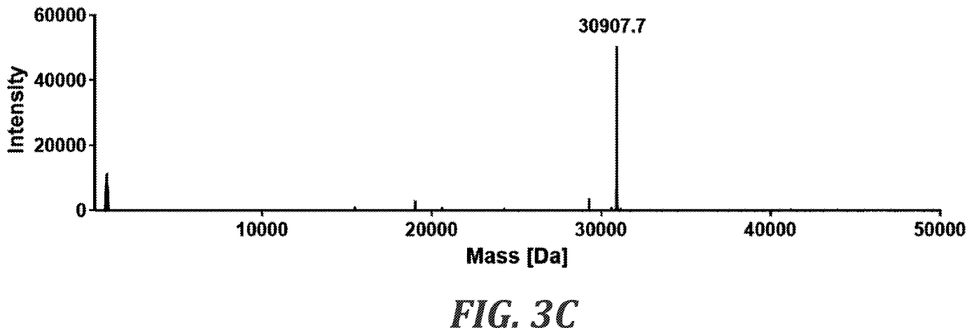

Protein verification of PXP (SEQ ID NO:81) is shown in FIGS. 3A-3C.

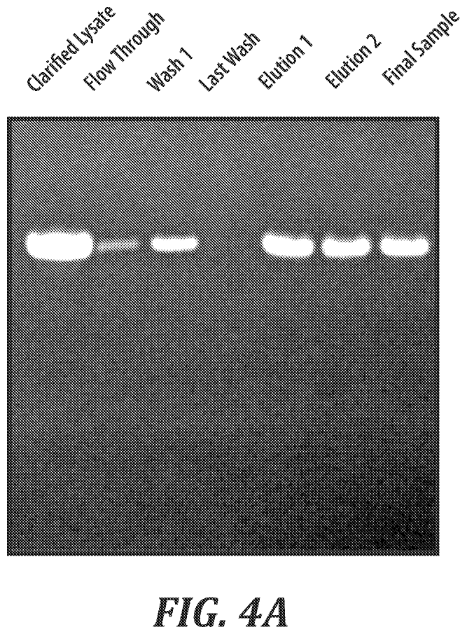

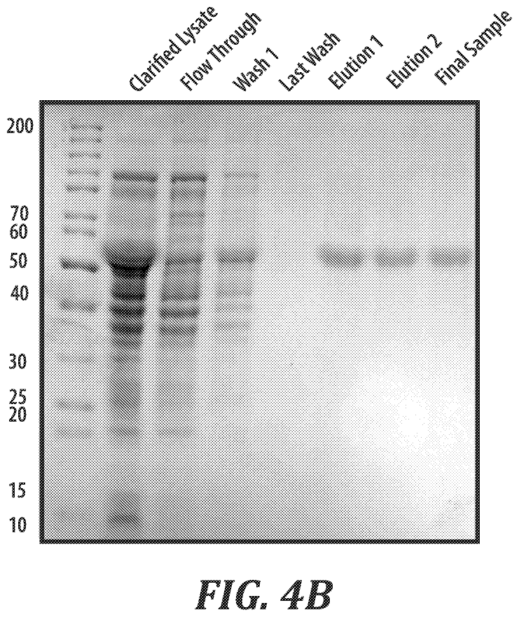

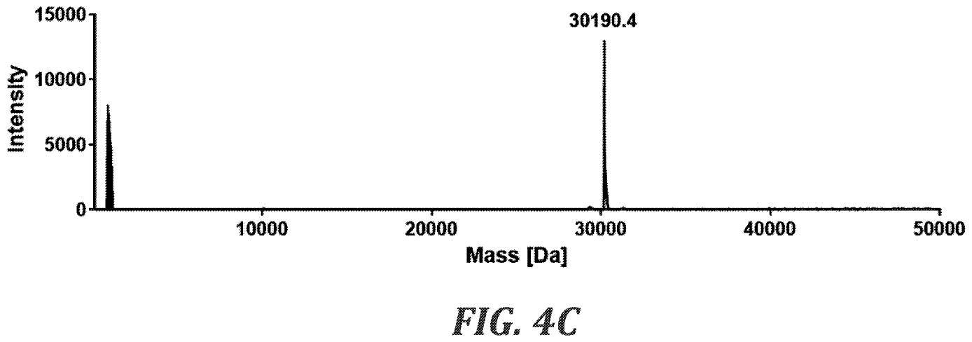

Protein verification of mutant T40A (SEQ ID NO:82) is shown in FIGS. 4A-4C.

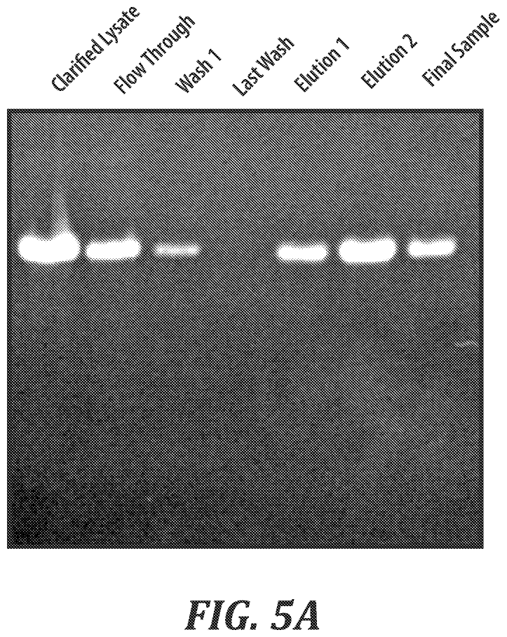

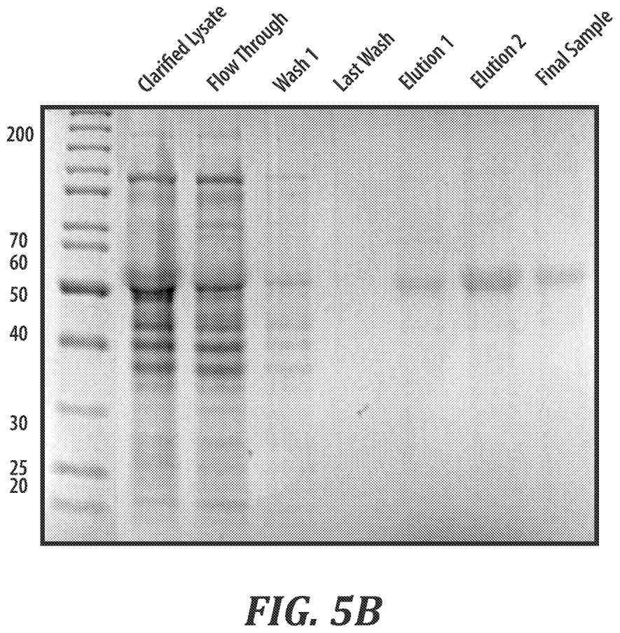

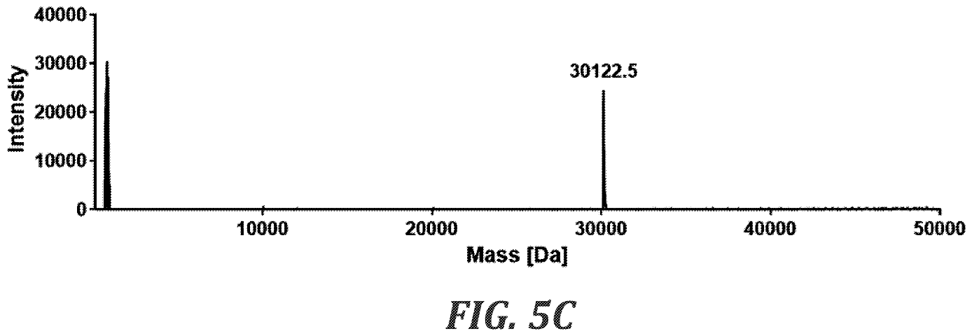

Protein verification of mutant Q54A (SEQ ID NO:83) is shown in FIGS. 5A-5C.

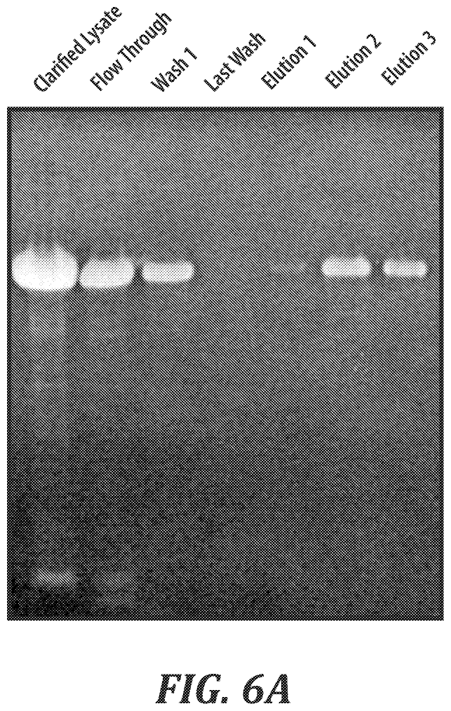

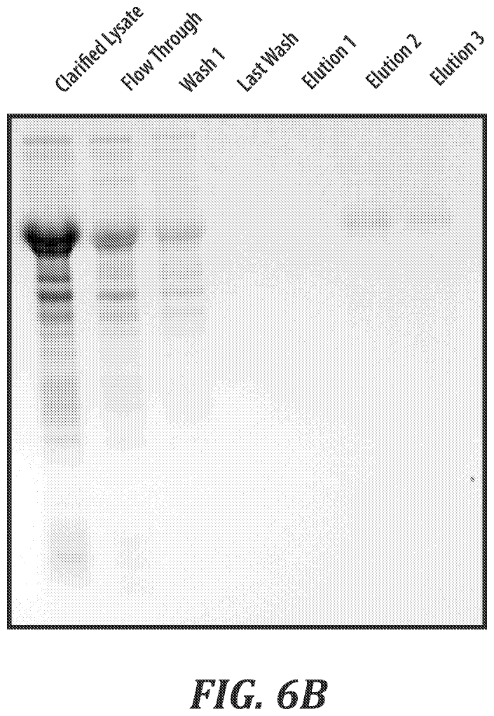

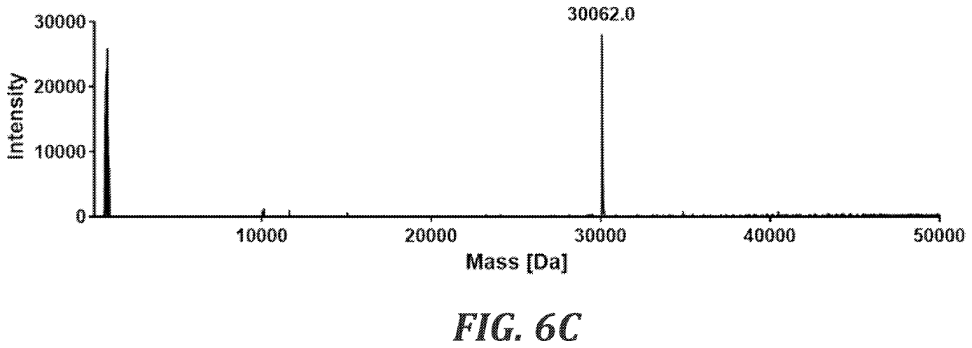

Protein verification of mutant T40A+Q54A (SEQ ID NO:84) is shown in FIGS. 6A-6C.

The foregoing demonstrate successful isolation of a single product from Ni-NTA purification, as evidenced by a single band in the elution lanes corresponding to an apparent molecular weight of 60 kilodaltons. The final product ran high due to weak binding to SDS caused by a lack of hydrophobic amino acids in XTEN.

### Example 5

### Hydrogel Preparation and Rheology

Following protein expression and purification, lyophilized protein was resuspended in phosphate-buffered saline (PBS, pH 7.4) at 10% weight/weight (w/w). Gels were vortexed, centrifuged, incubated at 37° C. for 10 min, and gently rocked at 4° C. overnight to encourage uniform gel formation. For cell encapsulation studies, lyophilized protein was rehydrated in cell suspension at 10% (w/w) and incubated for 1 hour at 37° C. (or until gels were uniform) and gently mixed by a pipette tip.

Characterization of material properties was performed using a Physica® MCR 301 Rheometer with a parallel plate geometry (8 mm plate diameter, 500 m gap) and a Peltier® plate for temperature control. Once the geometry reached the measurement position, mineral oil was applied to the surrounding edges of the gel to prevent evaporation.

The first segment included a 200 second (s) oscillatory time sweep at constant strain (5%) and frequency (30 radians per second (rad s⁻¹)) to ensure proper mixing and plateau storage modulus. Next, an angular frequency sweep was performed at constant strain (5%) with varied frequency (0.1-100 rad s⁻¹) to identify the linear viscoelastic range (LVER) followed by another time sweep to reset the gel. Then a strain sweep was implemented at constant frequency (30 rad s⁻¹) with varied strain (0-500%) to identify LVER followed by another time sweep to reset the gel. Subsequently, the cyclic strain sweep test was employed by toggling between low (5% strain, 30 min, within LVER) and high (500% strain, 1 min, outside of LVER) strain 4 times at constant angular frequency (30 rad s⁻¹, within LVER). Finally, a rotational shear thinning test at increasing shear rate (0.1-50 s⁻¹) was implemented to demonstrate decreasing viscosity with increasing shear. A total of five replicates were repeated for each hydrogel type (PXP, T40A, Q54A, and T40A+Q54A). All tests were performed at 25° C. (relevant for injection temperature) followed by 37° C. (body temperature).

Rheology data utilized Python® to calculate the average storage modulus, strain crossover, frequency crossover, and recovery time for each gel and condition. The storage modulus was calculated as the average of the last 25 data points in the first and second time sweeps. Strain and frequency crossovers were interpolated to determine when G″ (loss modulus)>G′ (storage modulus) during the corresponding strain and frequency sweep tests. Recovery time crossover was interpolated as the time it takes to recover back to the gel state (G′>G″) after periods of high strain during the cyclic strain sweep test. A multiple comparisons two-way ANOVA® table was applied for statistical analysis to determine significance between mutant types.

### Example 6

### Biodegradation

For biodegradation studies, 50 μL gels (N=3 per gel type) at 10% (w/w) were formed in the bottom of a 15 mL Falcon™ tube and washed twice with 5 mL PBS+0.75 mM phenylmethylsulfonyl fluoride (PMSF) to remove any initially unincorporated protein. 10 mL of fresh PBS containing 0.75 mM PMSF was replaced on top of the gel and the tubes were incubated at 37° C. for the remainder of the study. Time points were taken every 24 hrs by centrifugation of samples at 200×g for 1 minute followed by removal of 100 μL fluid for later analysis. To replace the fluid removed, 100 μL fresh PBS containing 0.75 mM PMSF was added back to each sample. The test continued for 32 days, at which point intact gel was still visible at the bottom of the tube.

Protein concentration in the supernatant at each time point was used to determine extent of gel erosion. A bicinchoninic acid (BCA) assay with a standard curve of known PXP concentrations ranging from 5-700 μg mL⁻¹ was utilized to quantify protein concentration in the samples. Samples were measured in technical duplicates on a 96-well plate, and fluorescence was detected (λₑₘᵢₛₛᵢₒₙ=562 nm) on a plate reader. Values were adjusted based on the total protein mass in each wash sample, the amount of protein removed at each time point, and evaporation in the tubes throughout the course of the study to obtain the final values for analysis.

### Example 7

### Human ESC-CM Injection Protection

hESC-CMs derived from RUES2 stem cells were resuspended at a concentration of 10 million cells within 100 μL of 10% (w/w) PXP gels and pro-survival cocktail (PSC) components (Pro-survival cocktail: Roswell Park Memorial Institute (RPMI)-based pro-survival cocktail for cell implantation containing 50% (vol/vol) growth factor-reduced Matrigel™, 100 μM ZVAD (benzyloxycarbonyl-Val-Ala-Asp(O-methyl)-fluoromethyl ketone), 50 nM Bcl-XL BH4 (cell-permeant TAT peptide), 200 nM cyclosporine A, 100 ng mL⁻¹ IGF-1, and 50 μM pinacidil). The injectate was pushed through a 26 G needle at 20 μL s⁻¹. As a control, another suspension of 10 million cells within a liquid-only PSC was also injected for comparison. Cell viability was analyzed directly after injection using an NC200™ NucleoCounter®. Final cell populations included technical duplicate with N=15 per sample group. A multiple comparisons one-way ANOVA® table was implemented for statistical analysis.

### Example 8

### 72-Hour Encapsulation of Fibroblasts and Human ESC-CM

NIH 3T3 Fibroblasts were suspended as 5 million cells per mL in Dulbecco's Modified Eagle Medium (DMEM) supplemented with 10% Fetal Bovine Serum and 1% Penicillin/Streptomycin. Human hESC-CMs were suspended as 10 million cells per mL in media (RPMI supplemented with 2% B-27 and 10% Penicillin/Streptomycin).

Cell suspensions (Fibroblast or hESC-CM) were added directly to lyophilized PXP protein resulting in 10% (w/w) gels. Encapsulated cells, and a cell-only control group, were injected in triplicate at a volume of 20 μL per well (384-well plate) through a 26 G needle. Two additional groups were added for the hESC-CM study, including a cell suspension in PSC (as described in injection protection methods) and cells encapsulated in PXP at 10% (w/w) with PSC. 20 μL of fresh media was added to each well, then the plate was incubated for 24 hrs at 37° C.

Cells were stained with 4 M CalceinAM (λₑₓₑᵢₜₐₜᵢₒₙ=488 nm, λₑₘᵢₛₛᵢₒₙ=520 nm) and 8 M Ethidium Homodimer-1 (λₑₓₑᵢₜₐₜᵢₒₙ=580 nm, λₑₘᵢₛₛᵢₒₙ=604 nm) then imaged on a Leica® Stellaris® 5 Confocal through the full thickness of each sample with a step size of 3 m in the z direction. Viability was quantified using the Leica® Application Suite X (LAS X) software by implementation of an Otsu threshold for both channels (CalceinAM and Ethidium Homodimer-1). Cells were incubated at 37° C. for an additional 48 hrs, and final cell viability of each well was analyzed using an NC200™ NucleoCounter®. A multiple comparisons one-way ANOVA® table was implemented for statistical analysis.

To model the incorporation of a protein of interest, PeGFP (eGFP with a single P domain) was added to the PXP gels. Lyophilized PXP was rehydrated with either PeGFP or eGFP (without a P domain) in phosphate buffer, both resulting in an eGFP molar concentration of 84 M throughout the gel (6.5% of the pentameric P domain interactions functionalized with a PeGFP). 50 μL gels (N=3) were formed in the bottom of 1.5 mL Eppendorf™ tubes at 10% (w/w) PXP and 500 μL phosphate buffer was added to the top of each. 2 μL fluid was removed every hour, diluted in 23 μL phosphate buffer, and fluorescence was monitored in a 384-well plate until a plateau concentration occurred (λₑₓₑᵢₜₐₜᵢₒₙ=488 nm, λₑₘᵢₛₛᵢₒₙ=530 nm). Fluorescence standard curves of known PeGFP and eGFP concentrations ranging from 0.02-5 M were employed to quantify eGFP released. Successful functionalization results in PeGFP released at a slower rate than eGFP without a P domain.

### Example 9

### Injectability and Recovery of Gels Results

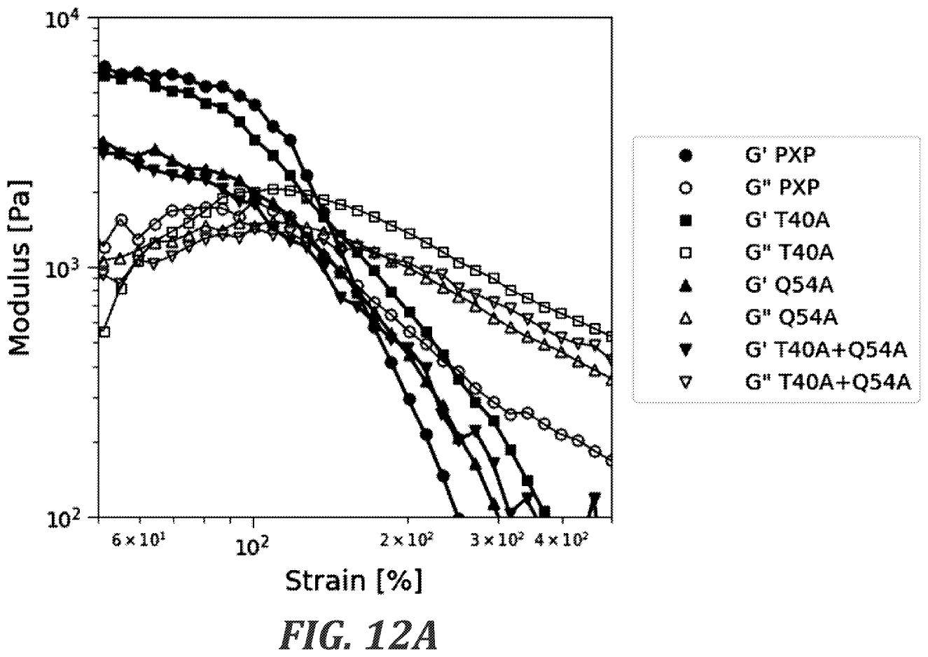

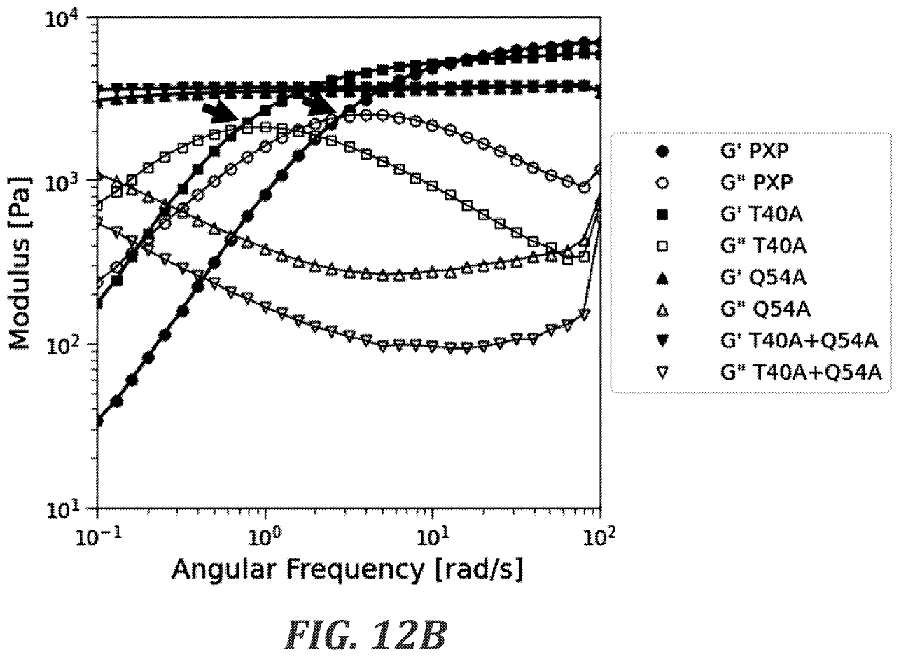

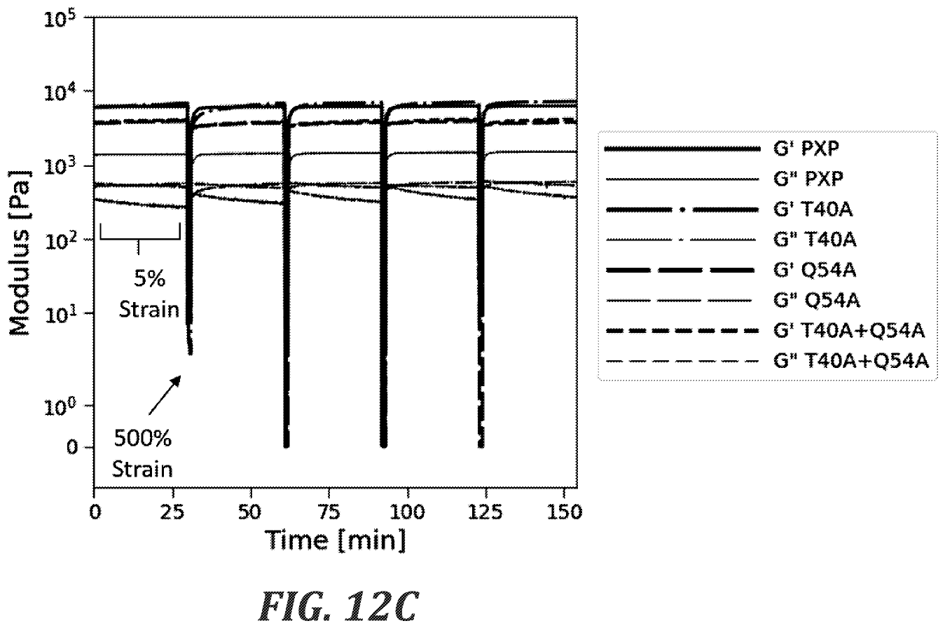

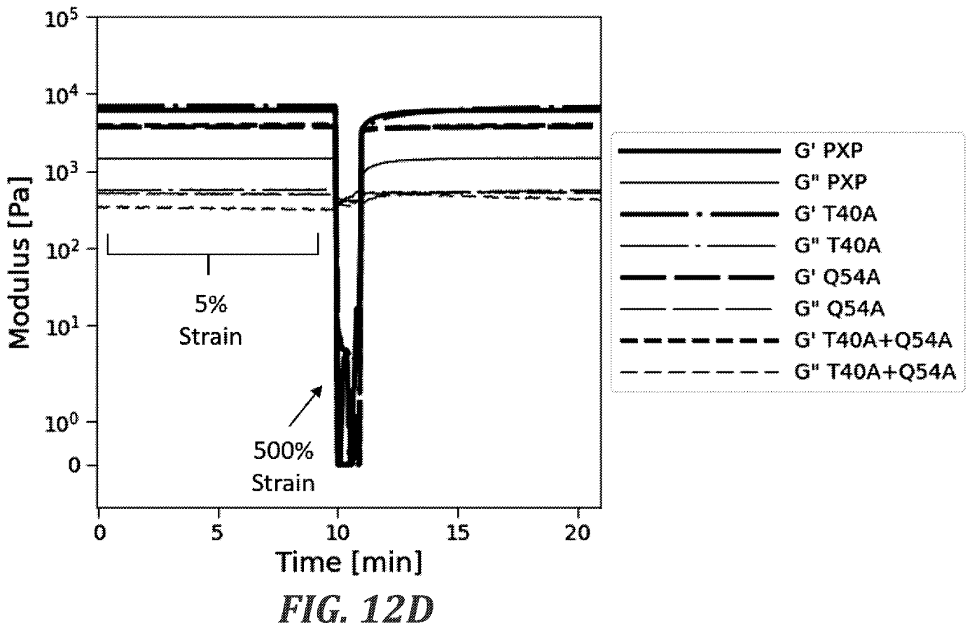

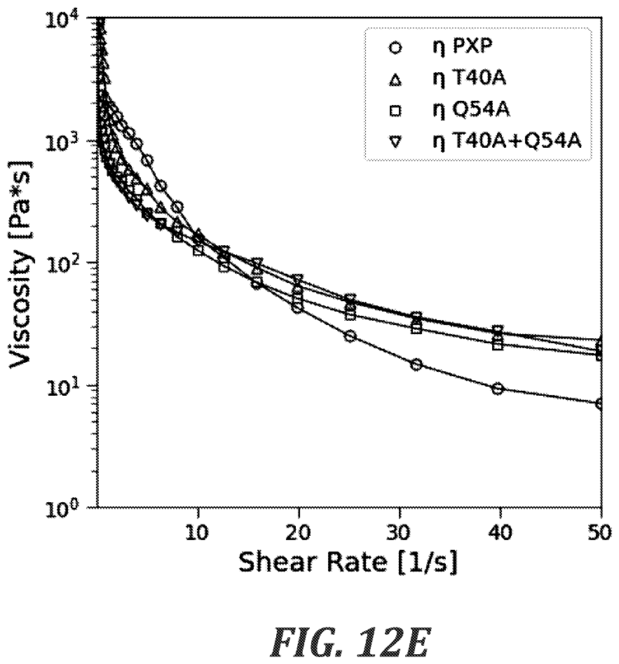

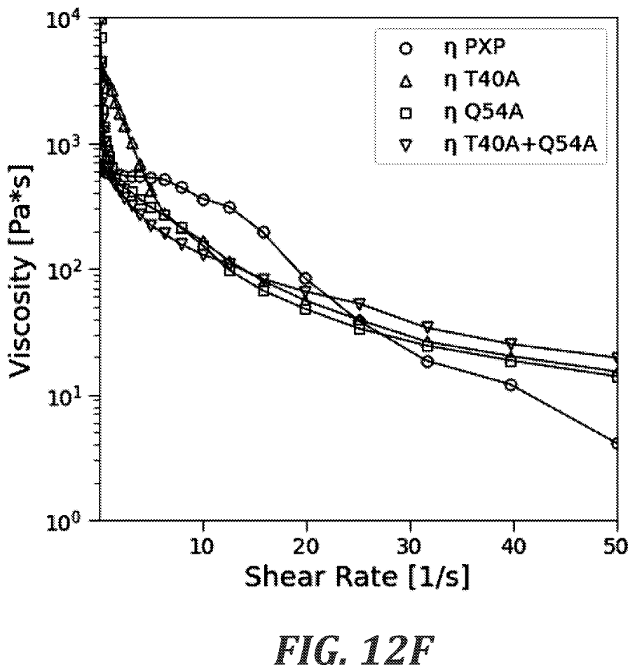

Purified recombinant proteins yielded stable hydrogels when reconstituted with PBS at 10% (w/w) and were macroscopically injectable through a 26 G needle at room temperature (25° C.) and physiologically relevant temperature (37° C.) (FIGS. 1D-1F, and FIGS. 12A-12F). The purified recombinant proteins exhibited shear-thinning and self-healing behavior in rheological assessment. The introduction of point mutations (T40A, Q54A, or both T40A+Q54A) stabilized the coiled-coil interactions by exchanging two polar residues for the smaller hydrophobic alanine to allow for improved association between the self-association domains.

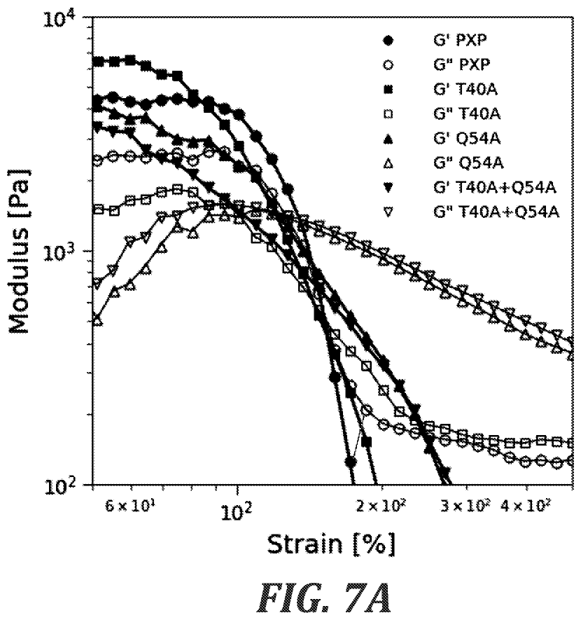

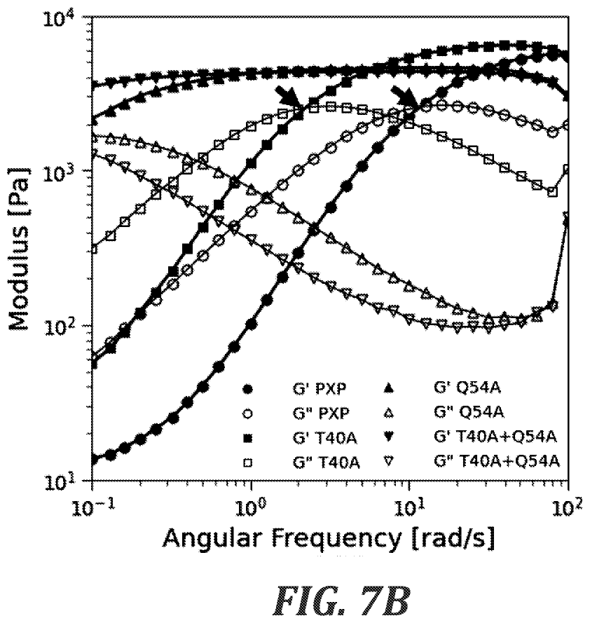

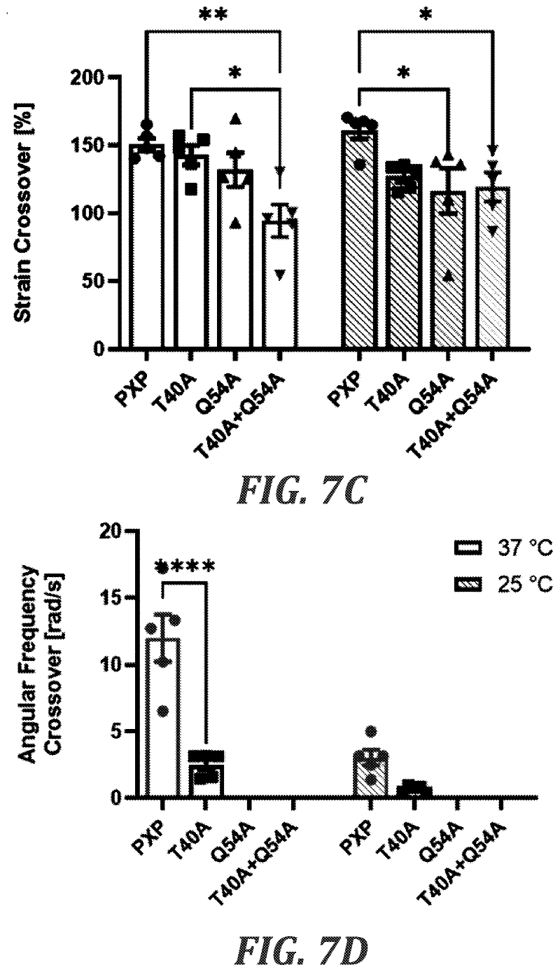

Strain sweeps identified a strain crossover for each gel type indicating the injectable nature of PXP and mutants by moving from an elastic material at low strain (G′>G″) to a viscous material at high strain (G′<G″) (FIG. 7A). The strain crossover at 37° C. confirmed breaking of physical coiled-coil bonds under high strain and occurs at 151±5% strain for PXP, 143±7% strain for T40A, 132±13% strain for Q54A, and 95±12% strain for T40A+Q54A, allowing for successful injection through a 26 G needle (Table 2). A statistically significant decrease (p<0.01) in strain crossover was observed with the introduction of both self-association domain mutations (T40A+Q54A), which deviated from the expectations for stabilizing coiled-coil interactions (FIG. 7C), but the strain crossover is sufficiently high for all PXP mutants to avoid strain-induced liquification through beating-associated heart contraction.

The frequency sweeps indicate that with increasing frequencies, elastic properties are favored over viscous properties (G′>G″), as there is minimal time for hydrogels to flow at higher frequencies. Frequency sweeps identified the angular frequency crossover to define the elastically favored region (in the gel state), which was extended with mutated self-association domains (FIG. 7B). No frequency crossover was found for Q54A or T40A+Q54A, indicating a larger working range of frequencies for these materials. In addition, the T40A crossover at 2.5±0.4 rad s⁻¹ was lower than the unmodified PXP at 12.0±1.8 rad s⁻¹, which extended the frequency LVER (37° C.) (FIG. 7D, Table 2). The human heart beats between 6.3-10.5 rad s⁻¹, meaning the best material selection would include the heart frequencies within the LVER, such as with Q54A or T40A+Q54A.

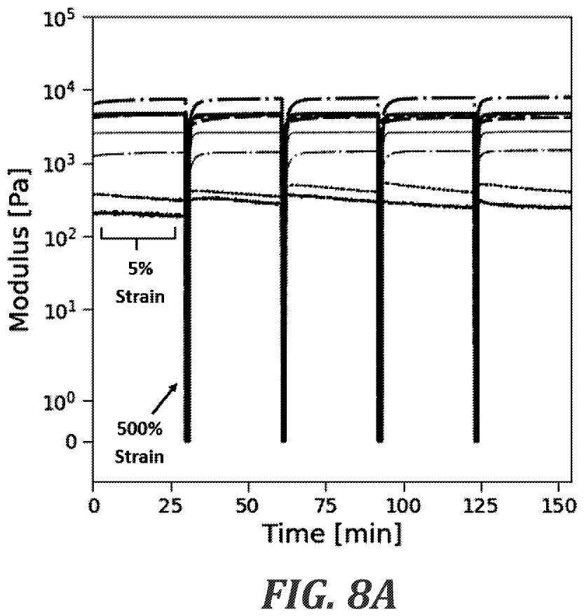

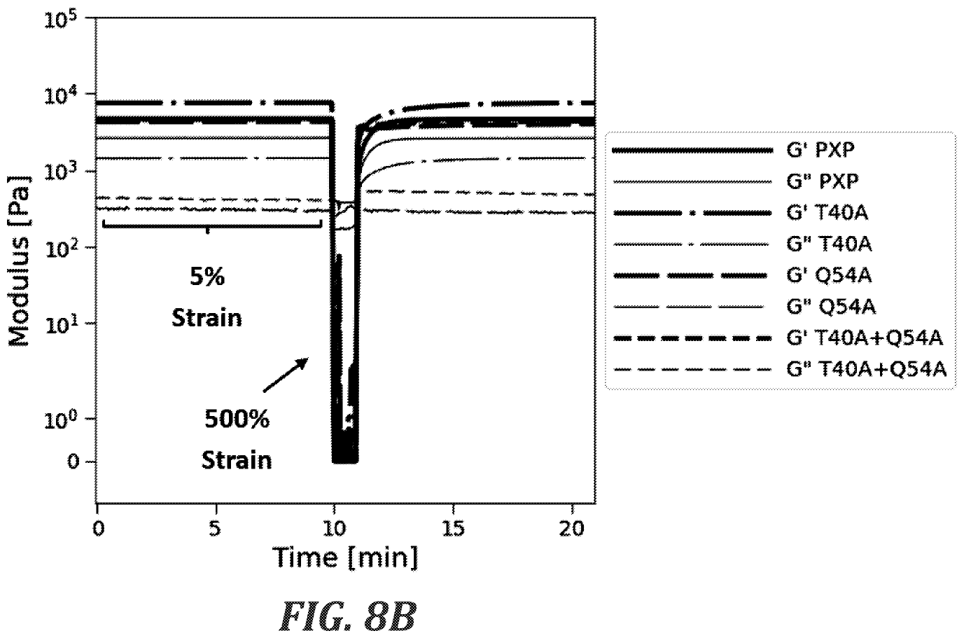

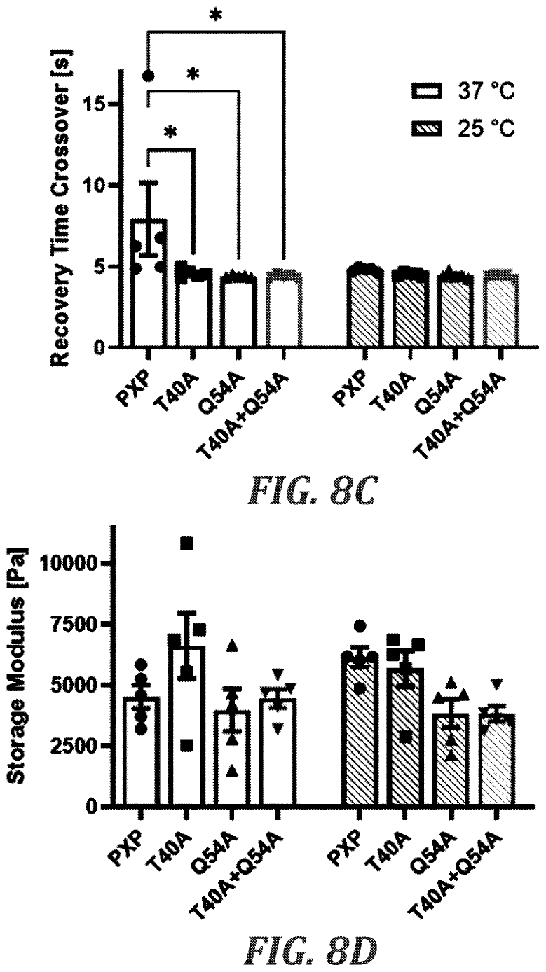

The storage modulus (G′) of PXP and mutants fall between 3.8-6.1 kilopascals (kPa) (PXP, 4.5±0.5 kPa; T40A, 6.6±1.3 kPa; Q54A, 4.0±0.9 kPa; T40A+Q54A, 4.5±0.4 kPa) during time sweeps at constant 5% strain and 30 rad s⁻¹ frequency, which is within range for materials that have been reported in the past for injectable heart cell therapies (approximately 0.5-5 kPa). No statistical significance was found between the storage moduli of mutants (FIG. 8D, Table 2).

Cyclic strain sweeps were employed to test the self-healing behavior and recovery time of each gel type. The operating frequency for cyclic strain sweep tests was within the LVER for all mutants at both temperatures (30 rad s⁻¹). The high strain value was well above the crossover strain for all mutants (500%), and the low strain was below crossover strain for all mutants (5%). Cyclic strain sweeps confirmed self-healing behavior for all gels, or the reforming of coiled-coil physical bonds upon return to low strain. Full gel recovery and minimal hysteresis (FIG. 8A) was observed after 4 cycles of high strain for all gel types and a recovery time of 7.91±2.23 s for PXP, 4.58±0.12 s for T40A, 4.39±0.04 s for Q54A, and 4.38±0.05 s for T40A+Q54A at 37° C. (FIG. 8B, FIG. 8C, Table 2).

A statistically significant (p<0.05) 3 s reduction in recovery time was identified from the introduction of point mutations due to highly favorable interactions of the stabilized mutated P domains.

TABLE 2
Summary of rheological properties for each gel at 25° C. and 37° C.

| | Temp | | | | |
|---|---|---|---|---|---|
| Parameter | [° C.] | PXP | T40A | Q54A | T40A + Q54A |
| Storage Modulus | 25 | 6.1 ± 0.4 | 5.7 ± 0.7 | 3.8 ± 0.6 | 3.8 ± 0.3 |
| [kPa] | 37 | 4.5 ± 0.5 | 6.6 ± 1.3 | 4.0 ± 0.9 | 4.5 ± 0.4 |
| | 25 | 161 ± 6 | 127 ± 4 | 116 ± 17 | 119 ± 11 |
| | 37 | 151 ± 5 | 143 ± 7 | 132 ± 13 | 95 ± 12 |
| Frequency | 25 | 3.0 ± 0.6 | 0.7 ± 0.1 | — | — |
| Crossover [rad s⁻¹] | 37 | 12.0 ± 1.8 | 2.5 ± 0.4 | — | — |
| Recovery Time | 25 | 4.79 ± 0.06 | 4.51 ± 0.05 | 4.45 ± 0.09 | 4.39 ± 0.06 |
| Crossover [s] | 37 | 7.91 ± 2.23 | 4.58 ± 0.12 | 4.39 ± 0.04 | 4.38 ± 0.05 |

Storage modulus calculated from time sweeps (5% strain, 30 rad s⁻¹). Strain crossover (G″ > G′) calculated from strain sweeps (30 rad s⁻¹, 0-500% strain). Frequency crossover calculated from frequency sweeps (5% strain, 0.1-100 rad s⁻¹, G′ > G″). No frequency crossover was observed for Q54A or T40A + Q54A. Recovery time crossover (G′ > G″ after high strain period) calculated from cyclic strain sweeps (30 rad s⁻¹, 5% low strain, 500% high strain). Error reported as SEM, N = 5.

### Example 10

### Tunable Biodegradation Properties

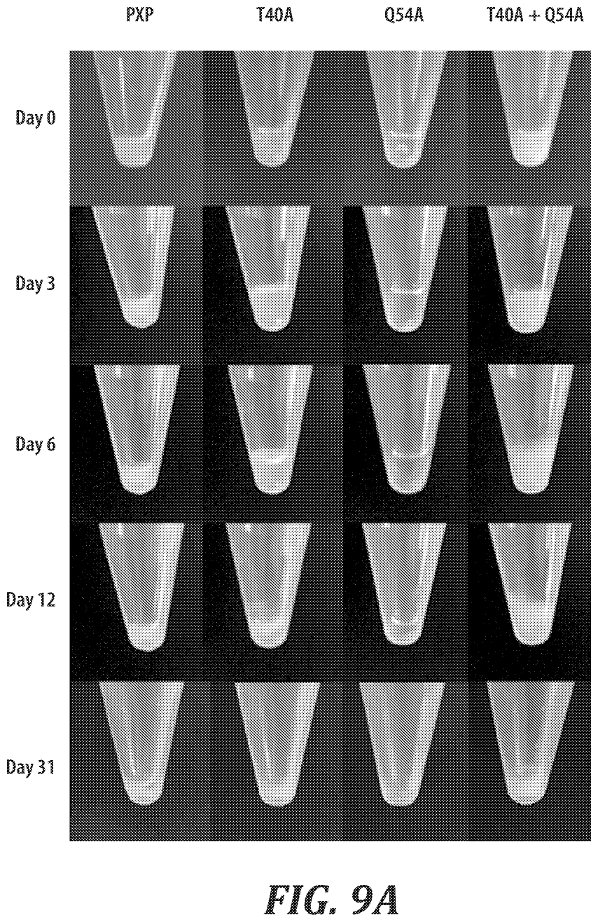

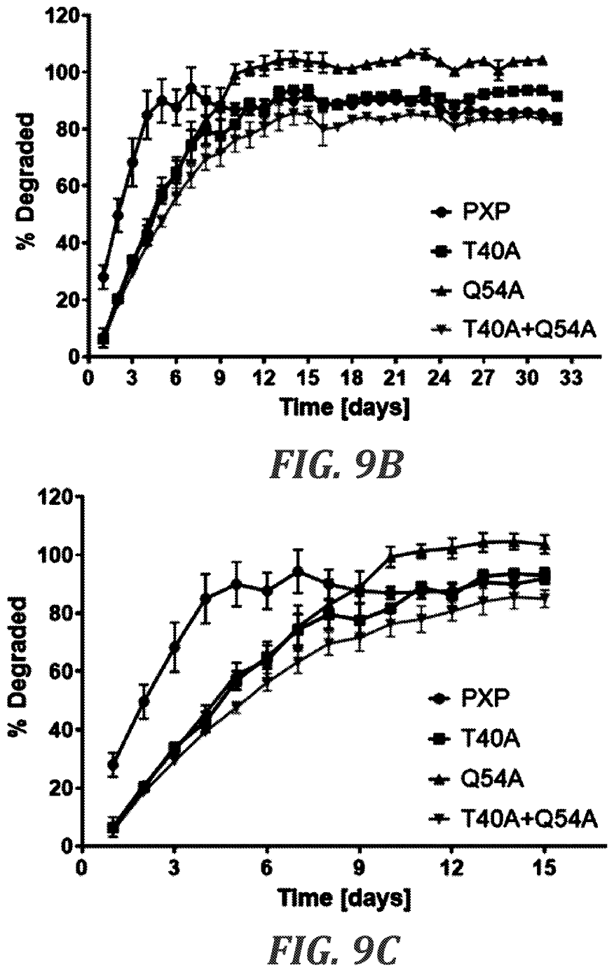

Biodegradation studies revealed gels incubated in PBS at 37° C. remain intact for up to 4 weeks, which helps injectable cell therapies, as cells need support during early engraftment. A slower degradation rate was observed during the first 11 days for all mutants (T40A, Q54A, and T40A+Q54A) when compared to PXP, confirming the mutations stabilized physical association between the self-association domains and allowing for tunable degradation profiles (FIGS. 9A-9C). The slowest erosion rate was observed in the double mutant (T40A+Q54A).

### Example 11

### Human ESC-CM Injection Protection and Cell Viability Post Injection

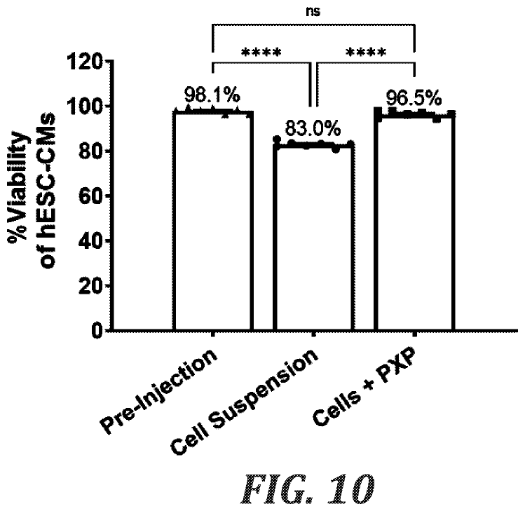

Encapsulating hESC-CM in PXP prior to injection improved cell survival by more than 10%, allowing for additional cells to be delivered to the heart (pre-injection: 98.1% viability, post injection liquid suspension: 83.0% viability, post injection in 10% (w/w) PXP: 96.5% viability) (FIG. 10). This demonstrates cell protection during the injection process by alleviating shear stress on the cells thus reducing disruption to cell membranes and providing a scaffold environment post injection. Improved cell survival post injection is crucial to increasing the efficiency of these injectable cell therapies and chance of engraftment to the host tissue. The results indicate PXP gels can be used as a delivery vehicle for injectable cardiomyocyte therapies.

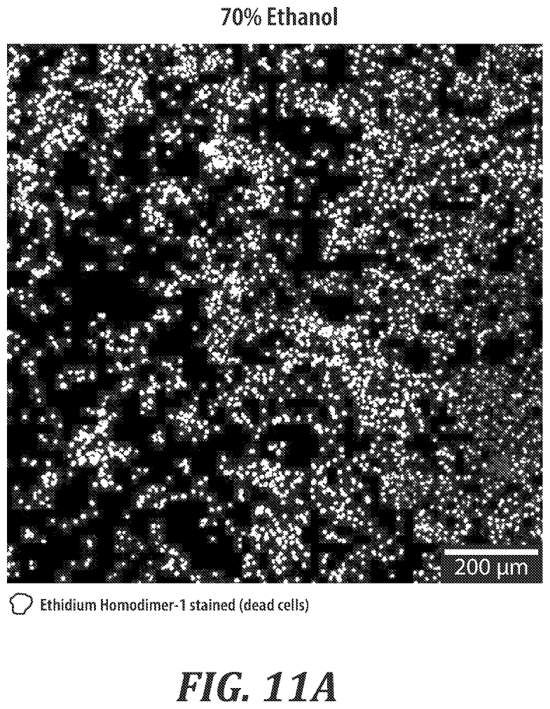

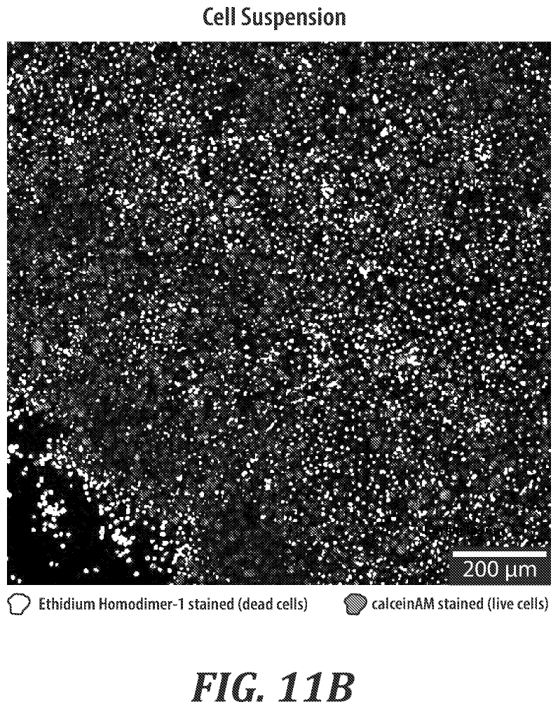

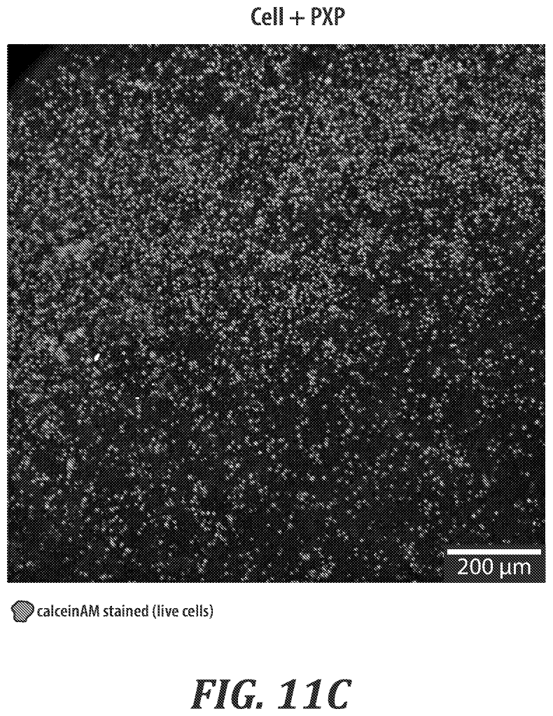

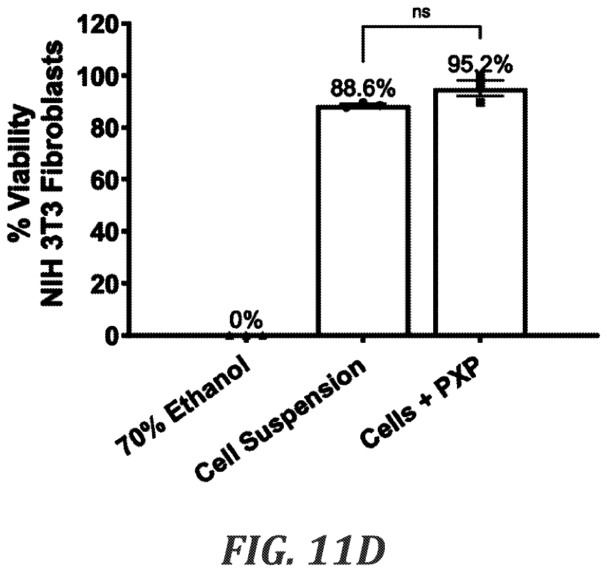

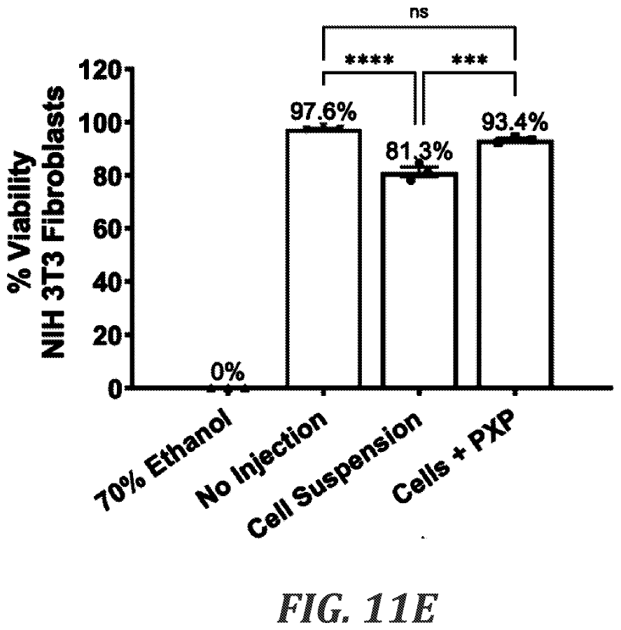

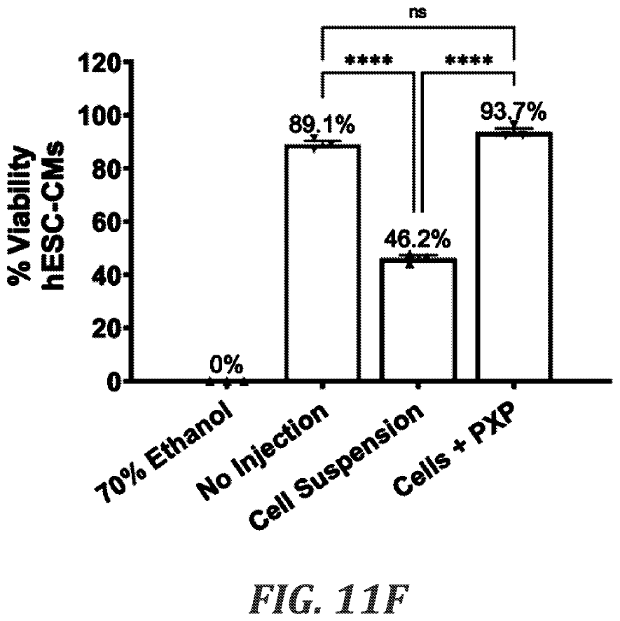

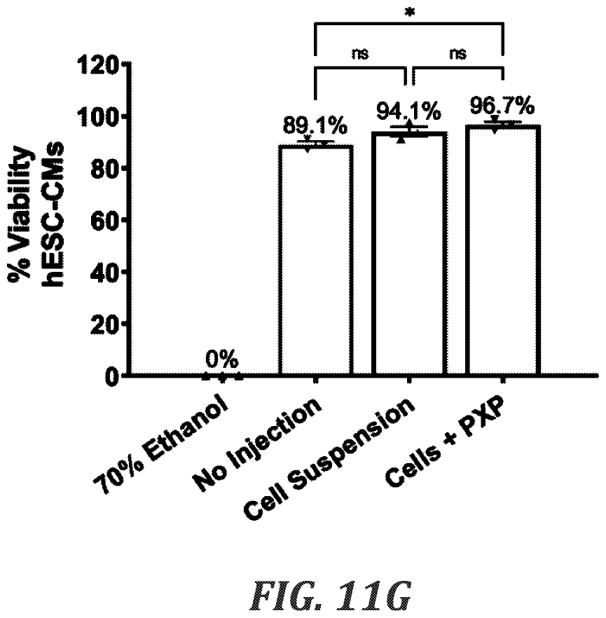

Improved cell viability was observed 24 hrs post-injection through a 26 G needle for NIH 3T3 fibroblasts encapsulated within PXP gels (88.6% viability) when compared to cell suspension alone (95.2% viability), as visualized by CalceinAM and Ethidium Homodimer-1 staining (FIGS. 11A-11D). Additionally, a continued high viability was observed (by NucleoCounter®) 72 hrs post injection with no statistical difference between fibroblasts injected within PXP (93.4% viability) and cells that were not injected (97.6% viability). Alternatively, a statistically significant decrease (p<0.001) in viability of cells that were injected in liquid suspension (81.3% viability) was observed (FIG. 11E).

The hESC-CMs also showed promising results 72 hrs post injection (FIG. 11F). No statistical significance was observed between pre-injection control (89.1% viability) and cells encapsulated in PXP (93.7% viability). However, a statistically significant decrease (p<0.0001) in viability with the cell suspension group (46.2% viability) was observed. High viability for all groups injected with the PSC (FIG. 11G) was observed. This indicates an added benefit that with the use of PXP, the PSC may no longer be necessary, which would reduce complications with FDA regulations and costs associated with PSC components. Fibroblasts and hESC-CMs showed improved viability for up to three days post injection when compared to a cell suspension group, demonstrating PXP cytocompatibility and offering 3D support to the cells.

### Example 12

### Biofunctionalization

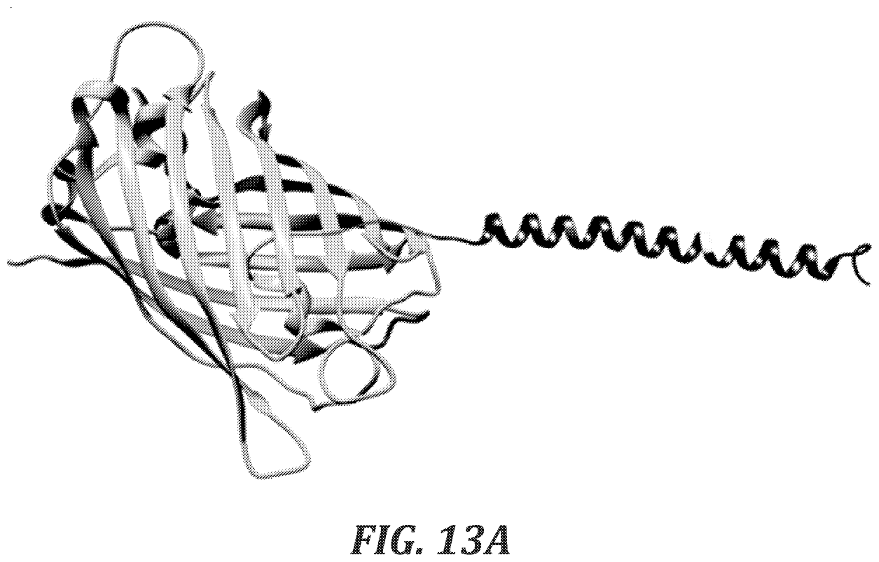

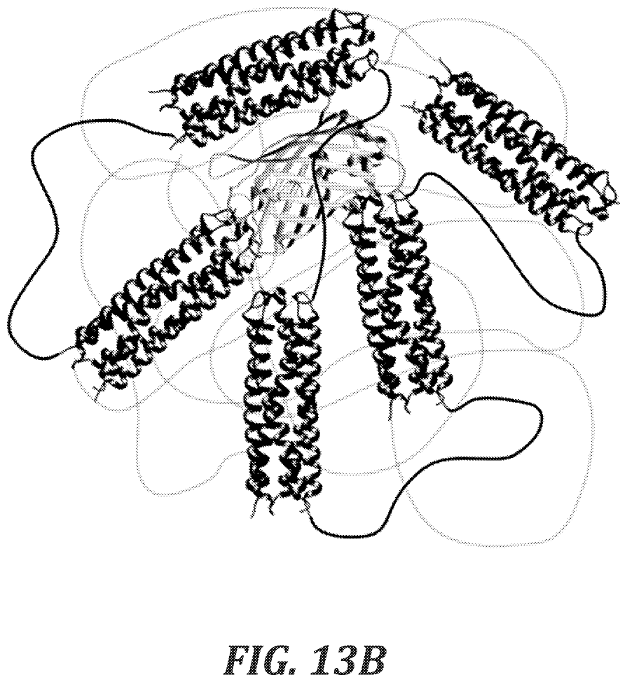

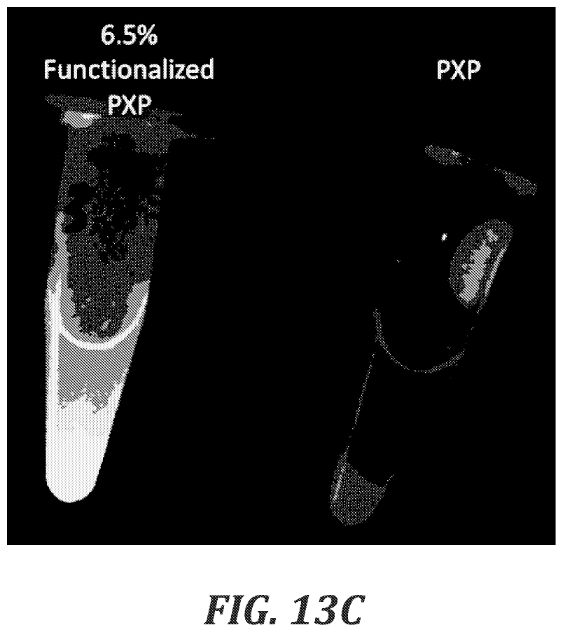

Therapeutic proteins including Delta-1 and erythropoietin were integrated into gel networks to promote desired cell outcomes and facilitate the healing process post myocardial infarction. To demonstrate that biofunctionalization of PXP gels could be readily attained using proteins site-specifically modified with a single P domain, eGFP was selected as a model protein of interest due to its ability to be easily tracked by fluorescence. Attaching a single P self-association domain to the model protein (FIGS. 13A and 13B) facilitates functionalization into the gel network. eGFP can be substituted for other user-defined therapeutic proteins.

PeGFP and eGFP were successfully expressed and purified. Fluorescence is observed in a 6.5% functionalized PXP gel, confirming that protein function is still intact with the addition of a single self-association P domain (FIG. 13C).

### Example 13

### Photodegradable Linker

A photodegradable recombinant protein hydrogel formed from a single protein, referred to herein as “PhoCoil,” was developed and characterized. This single-component, recombinant protein-based hydrogel is crosslinked by self-association domain interactions, exhibits shear-thinning and self-healing for easy injectability, and is selectively degradable in response to 405 nm light.

PhoCoil was constructed with self-association domains at each end of the recombinant protein, the self-association domains were connected to the flexible linker, and the photodegradable protein (PhoCl) was connected in between amino acids of the flexible linker amino acid sequence. The self-association domains formed physically associated homopentameric bundles that crosslink the proteins into the gel network of the hydrogel. PhoCl, a green fluorescent protein that undergoes irreversible cleavage of the peptide backbone in response to 405 nm light, enables the destruction of network crosslinks by externally controlled light. The unstructured flexible linker sequences reduce the steric hindrance to intermolecular interaction. A pET21 vector containing a gBlock (IDT) coding for PhoCoil was used to obtain the protein using common E. coli-based protein expression, and affinity chromatography, followed by lyophilization. PhoCoil was then resuspended in PBS or media to form gels characterized via rheometry, degradation studies, photolithographic response, and ability to support 3D encapsulation.

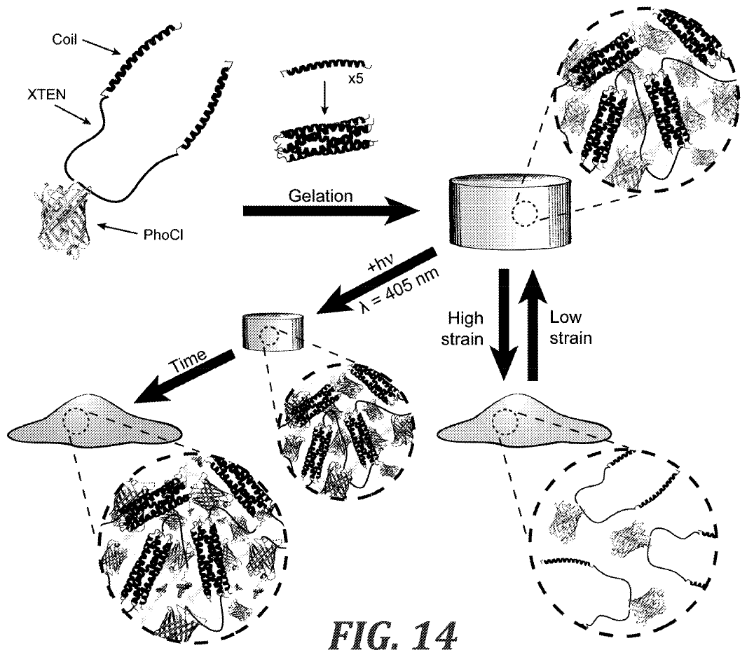

PhoCoil gel stiffness can be modulated by varying the protein weight percentage, forming gels from 1-4 kPa in storage modulus. All tested weight percentages showed a reduction in viscosity over three orders of magnitude in response to increasing shear rates, as well as a return to the original storage modulus after periods of high strain. Gel degradation rates also increased ˜10-fold after exposure to 405 nm light in comparison to gels kept in ambient light. Photorheometry studies, where controlled light is delivered directly to the gel during rheological measurements, showed that gels can be partially softened by controlling the duration of light exposure or by co-formulation with a non-light responsive self-associated domain protein network. Taking advantage of the ability to control light spatiotemporally, gel photopatterning was achieved at 10-micron resolution with gel degradation confined to user-defined areas that received light. Finally, photodegradation was achieved through thin tissue mimics. The cells were encapsulated, injected, cultured, and photoreleased from the PhoCoil gels, and were shown to retain high viability. A schematic of photodegradation linker incorporation and function is shown in FIG. 14.

While illustrative embodiments have been illustrated and described, it will be appreciated that various changes can be made therein without departing from the spirit and scope of the disclosure.

1. An inoculant composition, comprising:
a recombinant protein, a cell consortia, and a biocompatible medium,
wherein the recombinant protein comprises one or more self-association domains, and a flexible linker comprising a pseudorepeat of amino acids, and
wherein the recombinant protein contacts the cell consortia.

2. The inoculant composition of claim 1, wherein the flexible linker is intrinsically unstructured.

3. The inoculant composition of claim 1, wherein the pseudorepeat of the flexible linker consists of 5-7 different amino acids.

4. The inoculant composition of claim 1, wherein the flexible linker consists of alanine, glutamic acid, glycine, proline, serine, and threonine.

5. The inoculant composition of claim 1, wherein the flexible linker comprises a quantity of 36-1,728 amino acids.

6. (canceled)

7. The inoculant composition of claim 1, wherein the one or more self-association domains comprises a first self-association domain and a second self-association domain,
wherein the first self-association domain has a sequence that is the same or different from the second self-association domain.

8. (canceled)

9. The inoculant composition of claim 1, wherein each of the one or more self-association domains are structured, and wherein the structure comprises a coil.

10. The inoculant composition of claim 1, further comprising a cell adhesion motif at a first terminus of the recombinant protein, a cell adhesion motif at a second terminus of the recombinant protein, or a cell adhesion motif at both a first and a second terminus of the recombinant protein, wherein the cell adhesion motif of the first terminus and the cell adhesion motif of the second terminus are the same or are different.

11. (canceled)

12. (canceled)

13. The inoculant composition of claim 1, wherein the recombinant protein comprises a single strand.

14. The inoculant composition of claim 1, further comprising one to five additional recombinant proteins, wherein a sequence of a first or a second self-association domain of the recombinant protein is the same or is different from a sequence of a first or a second self-association domain of each of the one to five additional recombinant proteins.

15. The inoculant composition of claim 14, wherein the first self-association domain forms a reversible intramolecular interaction with the second self-association domain of the recombinant protein; the first self-association domain of the recombinant protein forms a reversible intermolecular interaction with the first or second self-association domain of each of the one to five additional recombinant proteins; the second self-association domain of the recombinant protein forms a reversible intermolecular interaction with the first or second self-association domain of the one to five additional recombinant proteins; or a combination thereof.

16. The inoculant composition of claim 14, wherein the recombinant protein of the inoculant composition interacts with four additional recombinant proteins to form a pentameric unit between the self-association domains, connected by intermolecular interactions, intramolecular interactions, or a combination thereof.

17. The inoculant composition of claim 1, further comprising a photodegradable linker.

18. (canceled)

19. The inoculant composition of claim 1, wherein the cell consortia comprises the same cells or different cells, and wherein the cells are selected from the group consisting of: fibroblasts, cardiomyocytes, stromal cells, neural cells, islet cells, embryonic stem cells, induced pluripotent stem cells, mesenchymal stem cells, neural cells, retinal cells, or a combination thereof.

20. The inoculant composition of claim 1, wherein a weight of the cell consortia is between about 2% and about 20% of a weight of the recombinant protein.

21. (canceled)

22. The inoculant composition of claim 1, wherein the recombinant protein is connected to a cell of the cell consortia by interacting with an antigen, a growth factor, a cell binding motif, a cytokine, an immunomodulator, or a combination thereof.

23. The inoculant composition of claim 1, wherein the recombinant protein forms a hydrogel, and wherein the cell consortia is encapsulated by the hydrogel.

24. The inoculant composition of claim 1, wherein the recombinant protein and cell consortia are disposed in the biocompatible medium,
wherein the biocompatible medium comprises water, one or more nutrients, one or more biocompatible constituents, or a combination thereof,
wherein the one or more nutrients comprises a buffer, salt, BcL-XL BH4, cyclosporin A, pinacidil, IGF-1, ZVAD, RPMI, DMEM, fetal bovine serum, bovine serum albumin, or a combination thereof, and
wherein the one or more biocompatible constituents comprises collagen, silk, resillin, gelatin, alginate, Matrigel, decellularized extracellular matrix, fibrin, PEG, zwitterionic polymer, PHEMA, PNIPAAM, hyaluronic acid, or a combination thereof.

25. The inoculant composition of claim 1, wherein the inoculant composition is fused to a cell of a biological tissue, wherein the cell of a biological tissue is a cell from a heart, a muscle, an eye, a brain, a pancreas, or a skin.

26. A method of cell therapy, the method comprising:
forming an inoculant composition, and
injecting the inoculant composition into a biological tissue,
wherein the inoculant composition comprises a recombinant protein, a cell consortia, and a biocompatible medium,
wherein the recombinant protein comprises one or more self-association domains and a flexible linker,
wherein the recombinant protein and cell consortia are disposed in the biocompatible medium, and
wherein the injecting causes shear thinning of the inoculant composition.

27-38. (canceled)


SEQUENCE LISTING (87 sequences)

SEQ ID NO: 1 - protein, 41 aa, synthetic construct
APQMLRELQETNAALQDVRELLRQQVKEITFLKNTVMESDA

SEQ ID NO: 2 - protein, 41 aa, synthetic construct
APQMLRELQEANAALQDVRELLRQQVKEITFLKNTVMESDA

SEQ ID NO: 3 - protein, 41 aa, synthetic construct
APQMLRELQETNAALQDVRELLRQAVKEITFLKNTVMESDA

SEQ ID NO: 4 - protein, 41 aa, synthetic construct
APQMLRELQEANAALQDVRELLRQAVKEITFLKNTVMESDA

SEQ ID NO: 5 - protein, 142 aa, synthetic construct
PAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAP

SEQ ID NO: 6 - protein, 5 aa, synthetic construct
GRGDS

SEQ ID NO: 7 - protein, 5 aa, synthetic construct
PHSRN

SEQ ID NO: 8 - protein, 21 aa, synthetic construct
DELPQLPHPNLHGPEILDVPS

SEQ ID NO: 9 - protein, 4 aa, synthetic construct
REDV

SEQ ID NO: 10 - protein, 19 aa, synthetic construct
YEKPGSPPREVVPRPRPGV

SEQ ID NO: 11 - protein, 15 aa, synthetic construct
KNNQKSEPLIGRKKT

SEQ ID NO: 12 - protein, 16 aa, synthetic construct
YRYRYTPKEKTGPMKE

SEQ ID NO: 13 - protein, 9 aa, synthetic construct
SPPRRARVT

SEQ ID NO: 14 - protein, 8 aa, synthetic construct
WQPPRARI

SEQ ID NO: 15 - protein, 6 aa, synthetic construct
KLDAPT

SEQ ID NO: 16 - protein, 7 aa, synthetic construct
TDIDAPS

SEQ ID NO: 17 - protein, 18 aa, synthetic construct
SRARKNAASIKVAVSADR

SEQ ID NO: 18 - protein, 4 aa, synthetic construct
RGDN

SEQ ID NO: 19 - protein, 17 aa, synthetic construct
KATPMLKMRTSFHGCIK

SEQ ID NO: 20 - protein, 21 aa, synthetic construct
KEGYKVRLDLNITLEFRTTSK

SEQ ID NO: 21 - protein, 21 aa, synthetic construct
KNLEISRSTFDLLRNSYGVRK

SEQ ID NO: 22 - protein, 17 aa, synthetic construct
DGKWHTVKTEYIKRKAF

SEQ ID NO: 23 - protein, 22 aa, synthetic construct
KQNCLSSRASFRGCVRNLRLSR

SEQ ID NO: 24 - protein, 12 aa, synthetic construct
NRWHSIYITRFG

SEQ ID NO: 25 - protein, 12 aa, synthetic construct
TWYKIAFQRNRK

SEQ ID NO: 26 - protein, 12 aa, synthetic construct
RKRLQVQLSIRT

SEQ ID NO: 27 - protein, 6 aa, synthetic construct
YIGSRC

SEQ ID NO: 28 - protein, 5 aa, synthetic construct
PDSGR

SEQ ID NO: 29 - protein, 21 aa, synthetic construct
RYVVLPRPVCFEKGKGMNYVR

SEQ ID NO: 30 - protein, 6 aa, synthetic construct
LGTIPG

SEQ ID NO: 31 - protein, 9 aa, synthetic construct
YGYYGDALR

SEQ ID NO: 32 - protein, 12 aa, synthetic construct
AFGVLALWGTRV

SEQ ID NO: 33 - protein, 12 aa, synthetic construct
DSITKYFQMSLE

SEQ ID NO: 34 - protein, 12 aa, synthetic construct
VILQQSAADIAR

SEQ ID NO: 35 - protein, 10 aa, synthetic construct
RNIAEIIKDI

SEQ ID NO: 36 - protein, 12 aa, synthetic construct
KAFDITYVRLKF

SEQ ID NO: 37 - protein, 13 aa, synthetic construct
TDIRVTLNRLNTF

SEQ ID NO: 38 - protein, 13 aa, synthetic construct
SETTVKYIFRLHE

SEQ ID NO: 39 - protein, 12 aa, synthetic construct
TSIKIRGTYSER

SEQ ID NO: 40 - protein, 4 aa, synthetic construct
RGDS

SEQ ID NO: 41 - protein, 4 aa, synthetic construct
RGDF

SEQ ID NO: 42 - protein, 12 aa, synthetic construct
HHLGGAKQAGDV

SEQ ID NO: 43 - protein, 7 aa, synthetic construct
KRLDGGS

SEQ ID NO: 44 - protein, 15 aa, synthetic construct
CQEPGGLVVPPTDAP

SEQ ID NO: 45 - protein, 15 aa, synthetic construct
LCDLAPEAPPPTLPP

SEQ ID NO: 46 - protein, 27 aa, synthetic construct
DLVFLLDGSSRLSEAEFEVLKAFVVDE

SEQ ID NO: 47 - protein, 4 aa, synthetic construct
RGDV

SEQ ID NO: 48 - protein, 9 aa, synthetic construct
SIGFRGDTC

SEQ ID NO: 49 - protein, 4 aa, synthetic construct
VTCG

SEQ ID NO: 50 - protein, 6 aa, synthetic construct
CSVTCG

SEQ ID NO: 51 - protein, 35 aa, synthetic construct
VDAVRTEKGFLLLASLRQMKKTRGTLLALERKDHS

SEQ ID NO: 52 - protein, 12 aa, synthetic construct
FQGVLQNVRFVF

SEQ ID NO: 53 - protein, 8 aa, synthetic construct
RFYVVMWK

SEQ ID NO: 54 - protein, 5 aa, synthetic construct
RGDTP

SEQ ID NO: 55 - protein, 6 aa, synthetic construct
SRGDTG

SEQ ID NO: 56 - protein, 4 aa, synthetic construct
DGEA

SEQ ID NO: 57 - protein, 15 aa, synthetic construct
GVKGDKGNPGWPGAP

SEQ ID NO: 58 - protein, 12 aa, synthetic construct
TAGSCLRKFSTM

SEQ ID NO: 59 - protein, 13 aa, synthetic construct
GEFYFDLRLKGDK

SEQ ID NO: 60 - protein, 19 aa, synthetic construct
CNYYSNSYSFWLASLNPER

SEQ ID NO: 61 - protein, 6 aa, synthetic construct
FYFDLR

SEQ ID NO: 62 - protein, 6 aa, synthetic construct
FTLCPR

SEQ ID NO: 63 - protein, 4 aa, synthetic construct
RHDS

SEQ ID NO: 64 - protein, 8 aa, synthetic construct
EPRGDNYR

SEQ ID NO: 65 - protein, 15 aa, synthetic construct
PSITWRGDGRDLQEL

SEQ ID NO: 66 - protein, 28 aa, synthetic construct
VLYGPRLDERDAPGNWTWPENSQQTPMC

SEQ ID NO: 67 - protein, 12 aa, synthetic construct
KELLLPGNNRKV

SEQ ID NO: 68 - protein, 11 aa, synthetic construct
PSKVILPRGGC

SEQ ID NO: 69 - protein, 9 aa, synthetic construct
CPCFLLGCC

SEQ ID NO: 70 - protein, 15 aa, synthetic construct
GKSFTIECRVPTVEP

SEQ ID NO: 71 - protein, 10 aa, synthetic construct
KYSFNYDGSE

SEQ ID NO: 72 - protein, 5 aa, synthetic construct
QIDSP

SEQ ID NO: 73 - protein, 4 aa, synthetic construct
IDSP

SEQ ID NO: 74 - protein, 4 aa, synthetic construct
GNEH

SEQ ID NO: 75 - protein, 4 aa, synthetic construct
KLEK

SEQ ID NO: 76 - protein, 10 aa, synthetic construct
LRAHAVDVNG

SEQ ID NO: 77 - protein, 7 aa, synthetic construct
YSDNGTF

SEQ ID NO: 78 - protein, 12 aa, synthetic construct
PPRWGLRNRPIN

SEQ ID NO: 79 - protein, 5 aa, synthetic construct
GRYDS

SEQ ID NO: 80 - protein, 307 aa, synthetic construct
MRGSHHHHHHGSVDGSGSGSGSGSGSGAPQMLRELQETNAALQDVRELLRQQVKEITFLKNTVMESDASGSGSGSGSGSGSLDGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPLDGSGSGSGSGSGSGAPQMLRELQETNAALQDVRELLRQQVKEITFLKNTVMESDASGSGSGSGSGSGSLEHHHHHHKLN

SEQ ID NO: 81 - protein, 317 aa, synthetic construct
MRGSHHHHHHGSGRGDSVDGSGSGSGSGSGSGAPQMLRELQETNAALQDVRELLRQQVKEITFLKNTVMESDASGSGSGSGSGSGSLDGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPLDGSGSGSGSGSGSGAPQMLRELQETNAALQDVRELLRQQVKEITFLKNTVMESDASGSGSGSGSGSGSLEGRGDSHHHHHHKLN

SEQ ID NO: 82 - protein, 310 aa, synthetic construct
MRGSHHHHHHGSGRGDSGSGSGSGSGSGSGAPQMLRELQEANAALQDVRELLRQQVKEITFLKNTVMESDASGSGSGSGSGSVDGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPLESGSGSGSGSGSGAPQMLRELQEANAALQDVRELLRQQVKEITFLKNTVMESDASGSGSGSGSGSGSGRGDSHHHHHHKLN

SEQ ID NO: 83 - protein, 310 aa, synthetic construct
MRGSHHHHHHGSGRGDSGSGSGSGSGSGSGAPQMLRELQETNAALQDVRELLRQAVKEITFLKNTVMESDASGSGSGSGSGSVDGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPLESGSGSGSGSGSGAPQMLRELQETNAALQDVRELLRQAVKEITFLKNTVMESDASGSGSGSGSGSGSGRGDSHHHHHHKLN

SEQ ID NO: 84 - protein, 310 aa, synthetic construct
MRGSHHHHHHGSGRGDSGSGSGSGSGSGSGAPQMLRELQEANAALQDVRELLRQAVKEITFLKNTVMESDASGSGSGSGSGSVDGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPLESGSGSGSGSGSGAPQMLRELQEANAALQDVRELLRQAVKEITFLKNTVMESDASGSGSGSGSGSGSGRGDSHHHHHHKLN

SEQ ID NO: 85 - protein, 6 aa, synthetic construct
HHHHHH

SEQ ID NO: 86 - protein, 320 aa, synthetic construct
MRGSHHHHHHGSGSGSGSGSGSGSGAPQMLRELQEANAALQDVRELLRQAVKEITFLKNTVMESDASGSGSGSGSGSVDMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKL

SEQ ID NO: 87 - protein, 265 aa, synthetic construct
MRGSHHHHHHGSGSGSGSGSGSVDMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKL